(19) United States
(12) Patent Application Publication
(10) Pub. No.: US 2022/0024851 A1
(43) Pub. Date: Jan. 27, 2022

(54) CRYSTALLINE FORMS OF MULTICYCLIC COMPOUNDS AND USES THEREOF

(71) Applicant: Convelo Therapeutics, Inc., Cleveland, OH (US)

(72) Inventors: Bradley T. Lang, North Olmsted, OH (US); Emily M. Rigsbee, West Lafayette, IN (US); Ekaterina V. Albert, West Lafayette, IN (US)

(21) Appl. No.: 17/299,463

(22) PCT Filed: Dec. 2, 2019
(86) PCT No.: PCT/US19/63963

§ 371 Date: Jun. 3, 2021

Related U.S. Application Data
(60) Provisional application No. 62/774,636, filed on Dec. 3, 2018.

(51) Int. Cl. C07C 211/40; C07C 309/04; C07C 65/10; A61K 45/06; A61K 31/136; A61K 31/192; A61P 25/28

(52) U.S. Cl. CPC C07C 211/40; C07C 309/04; C07C 65/10; C07B 2200/13; A61K 31/136; A61K 31/192; A61P 25/28; A61K 45/06

(57) ABSTRACT

The invention is directed at esylate and salicylate salts of cis-N-cyclohexyl-N-ethyl-3-(3-chloro-4-cyclohexylphenyl)prop-2-enylamine and their pharmaceutical composition. The invention is also directed at the use esylate and salicylate salts of cis-N-cyclohexyl-N-ethyl-3-(3-chloro-4-yclohexylphenyl)prop-2-enylamine and their pharmaceutical composition in treatment of myelin-related disorders.

### RELATED APPLICATIONS

This application claims the benefit of U.S. Provisional Application No. 62/774,636, filed on 3 Dec. 2018. The entire teachings of the above application are incorporated herein by reference.

### BACKGROUND

Myelin-related disorders are disorders that result in abnormalities of the myelin sheath (e.g., dysmyelination, demyelination and hypomyelination) in a subject's neural cells, e.g., CNS neurons including their axons. Degradation of the myelin sheath in such disorders, produces a slowing or cessation of nerve cell conduction. The resulting myelin related disorders are characterized by deficits in sensation, motor function, cognition, or other physiological functions. Myelin related disorders include, but are not limited to, multiple sclerosis (MS), neuromyelitis optica (NMO), optic neuritis, pediatric leukodystrophies, neonatal white matter injury, age-related dementia, schizophrenia, progressive multifocal leukoencephalopathy (PML), encephalomyelitis (EPL), central pontine myelolysis (CPM), adrenoleukodystrophy, Alexander's disease, Pelizaeus Merzbacher disease (PMD), Vanishing White Matter Disease, Wallerian Degeneration, transverse myelitis, amylotrophic lateral sclerosis (ALS), Huntington's disease, Alzheimer's disease, Parkinson's disease, spinal cord injury, traumatic brain injury, post radiation injury, neurologic complications of chemotherapy, stroke, acute ischemic optic neuropathy, vitamin E deficiency, isolated vitamin E deficiency syndrome, Bassen-Kornzweig syndrome, Marchiafava-Bignami syndrome, metachromatic leukodystrophy, trigeminal neuralgia, acute disseminated encephalitis, Guillian-Barre syndrome, Charcot-Marie-Tooth disease, Bell's palsy and radiation-induced demyelination.

MS is the most common myelin-related disorder affecting several million people globally and is estimated to result in about 18,000 deaths per year. MS is a complex neurological disease characterized by deterioration of central nervous system (CNS) myelin. Myelin, composed in its majority by lipids (70% lipids, 30% protein), protects axons and makes saltatory conduction possible, which speeds axonal electric impulse. Demyelination of axons in chronic MS can result in axon degeneration and neuronal cell death. Additionally, MS destroys oligodendrocytes, the highly specialized CNS cells that generate and maintain myelin. A repair process, called remyelination, takes place in early phases of the disease, but the oligodendrocytes are unable to completely rebuild the cell's myelin sheath. Repeated attacks lead to successively less effective remyelinations, until a scar-like plaque is built up around the damaged axons. These scars are the origin of the symptoms.

Several phenotypes (commonly termed types), or patterns of progression, have been described. Phenotypes use the past course of the disease in an attempt to predict the future course. They are important not only for prognosis but also for treatment decisions. Currently, the United States National Multiple Sclerosis Society and the Multiple Sclerosis International Federation, describes four types of MS (revised in 2013):

1. Clinically isolated syndrome (CIS)

### 2. Relapsing-remitting MS (RRMS)

3. Primary progressive MS (PPMS)
4. Secondary progressive MS (SPMS)

Relapsing-remitting multiple sclerosis is characterized by unpredictable relapses followed by periods of months to years of relative quiet (remission) with no new signs of disease activity. Deficits that occur during attacks may either resolve or leave problems, the latter in about 40% of attacks and being more common the longer a person has had the disease. This describes the initial course of 80% of individuals with multiple sclerosis. The relapsing-remitting subtype usually begins with a clinically isolated syndrome (CIS). In CIS, a person has an attack suggestive of demyelination, but does not fulfill the criteria for multiple sclerosis. 30 to 70% of persons experiencing CIS later develop multiple sclerosis.

Primary progressive multiple sclerosis occurs in approximately 10-20% of individuals, with no remission after the initial symptoms. It is characterized by progression of disability from onset, with no, or only occasional and minor, remissions and improvements. The usual age of onset for the primary progressive subtype is later than of the relapsing-remitting subtype. It is similar to the age that secondary progressive usually begins in relapsing-remitting multiple sclerosis, around 40 years of age.

Secondary progressive multiple sclerosis occurs in around 65% of those with initial relapsing-remitting multiple sclerosis, who eventually have progressive neurologic decline between acute attacks without any definite periods of remission. Occasional relapses and minor remissions may appear. The most common length of time between disease onset and conversion from relapsing-remitting to secondary progressive multiple sclerosis is 19 years.

At present, there is no cure for myelin-related disorders. Accordingly, there is a need for new therapeutic approaches to the treatment of myelin-related disorders, including the promotion of myelination.

### SUMMARY OF THE INVENTION

The present invention relates to pharmaceutically acceptable salts of cis-N-cyclohexyl-N-ethyl-3-(3-chloro-4-cyclohexylphenyl)prop-2-enylamine, represented by formula (I), or solvates thereof:

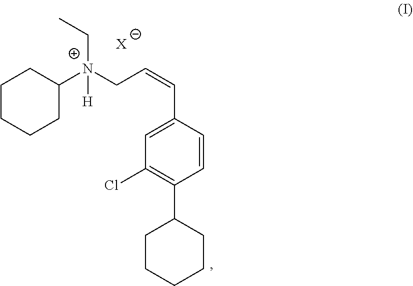

wherein:
X⁻ is selected from the group consisting of the anions of the following acids: ethanesulfonic acid (ESA), salicylic acid, adipic acid, benzenesulfonic acid (BSA), trans-cinnamic acid, ethanedisulfonic acid (EDSA), gentisic acid, glycolic acid, α-ketoglutaric acid, malic acid, malonic acid, 1,5-naphthalenedisulfonic acid (1,5-NDSA), L-tartaric acid, vanillic acid, and xinafoic acid.

In some embodiments, the present invention relates to a solid form of the compound represented by formula (II):

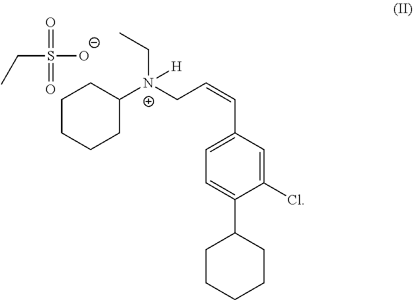

In certain embodiments the present invention relates to a solid form of the compound represented by formula (II), wherein the solid form is an amorphous, polymorphous, crystalline, or partially crystalline solid form.

In some embodiments the present invention relates to a solid form of the compound represented by formula (II), wherein the solid form is characterized by at least three x-ray powder diffraction peaks at 2θ angles selected from 12.50°, 16.73°, 17.87°, and 20.47°. Additionally, the present invention relates to a solid form of the compound represented by formula (II), wherein the solid form is characterized by at least four x-ray powder diffraction peaks at 20 angles selected from 12.50°, 14.00°, 16.73°, 17.87°, 18.65°, and 20.47°. In some embodiments the present invention relates to a solid form of the compound represented by formula (II), wherein the solid form is characterized by at least five x-ray powder diffraction peaks at 20 angles selected from 6.97°, 12.50°, 14.00°, 14.75°, 16.73°, 17.87°, 18.65°, 18.98°, and 20.47°.

In some embodiments the present invention relates to a solid form of the compound represented by formula (II), wherein the solid form is characterized by x-ray powder diffraction peaks at 2θ angles of 12.50°, 16.73°, 17.87°, and 20.47°. Additionally, the present invention relates to a solid form of the compound represented by formula (II), wherein the solid form is characterized by x-ray powder diffraction peaks at 2θ angles of 12.50°, 14.00°, 16.73°, 17.87°, 18.65°, and 20.47°. In certain embodiments the present invention relates to a solid form of the compound represented by formula (II), wherein the solid form is characterized by x-ray powder diffraction peaks at 2θ angles 6.97°, 12.50°, 14.00°, 14.75°, 16.73°, 17.87°, 18.65°, 18.98°, and 20.47°.

In some embodiments the present invention relates to a solid form of the compound represented by formula (II), wherein the solid form is characterized by an x-ray powder diffraction pattern substantially in accordance with that depicted in FIG. 1. Additionally, the present invention relates to a solid form of the compound represented by formula (II), wherein the solid form is characterized by a DSC thermogram having an endothermic event at about 137° C. In certain embodiments the present invention relates to a solid form of the compound represented by formula (II), wherein the solid form is characterized by a DSC thermogram substantially in accordance with that depicted in FIG. 3.

In some embodiments the present invention relates to a solid form of the compound represented by formula (II), wherein the solid form is substantially free of solvent, for example, substantially free of water. In certain embodiments the present invention relates to a solid form of the compound represented by formula (II), wherein the solid form is anhydrous.

In some embodiments the present invention relates to a solid form of the compound represented by formula (II), wherein the solid form is substantially pure, for example substantially free of chemical impurities, or substantially free of physical impurities.

In some embodiments the present invention relates to a solid form of the compound represented by formula (II), wherein the solid form is a solvate, for example a hydrate.

In certain embodiments the present invention relates to pharmaceutical composition comprising a solid form of the compound represented by formula (II) and a pharmaceutically acceptable carrier.

In some embodiments, the present invention relates to a solid form of the compound represented by formula (III):

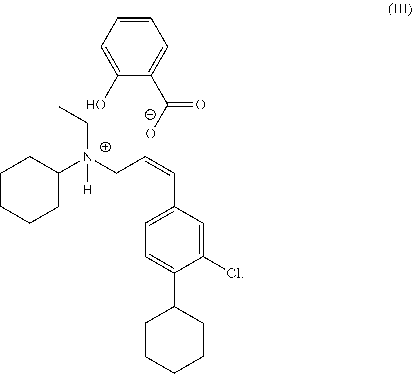 [SMILES: CC[NH+](C/C=C\c1ccc(C2CCCCC2)c(Cl)c1)C1CCCCC1 ; O=C([O-])c1ccccc1O]

In certain embodiments the present invention relates to a solid form of the compound represented by formula (III), wherein the solid form is an amorphous, polymorphous, crystalline, or partially crystalline solid form.

In some embodiments the present invention relates to a solid form of the compound represented by formula (III), wherein the solid form is characterized by at least three x-ray powder diffraction peaks at 2θ angles selected from 4.93°, 14.71°, 20.85°, and 25.39°. Additionally, the present invention relates to a solid form of the compound represented by formula (III), wherein the solid form is characterized by at least four x-ray powder diffraction peaks at 2θ angles selected from 4.93°, 14.71°, 19.03°, 20.85°, 24.94°, and 25.39°. In some embodiments the present invention relates to a solid form of the compound represented by formula (III), wherein the solid form is characterized by at least five x-ray powder diffraction peaks at 2θ angles selected from 4.93°, 12.79°, 14.71°, 16.97°, 17.59°, 19.03°, 20.85°, 22.08°, 24.94°, and 25.39°.

In some embodiments the present invention relates to a solid form of the compound represented by formula (III), wherein the solid form is characterized by x-ray powder diffraction peaks at 2θ angles of 4.93°, 14.71°, 20.85°, and 25.39°. Additionally, the present invention relates to a solid form of the compound represented by formula (I), wherein the solid form is characterized by x-ray powder diffraction peaks at 2θ angles of 4.93°, 14.71°, 19.03°, 20.85°, 24.94°, and 25.39°. In certain embodiments the present invention relates to a solid form of the compound represented by formula (I), wherein the solid form is characterized by x-ray powder diffraction peaks at 2θ angles 4.93°, 12.79°, 14.71°, 16.97°, 17.59°, 19.03°, 20.85°, 22.08°, 24.94°, and 25.39°.

In some embodiments the present invention relates to a solid form of the compound represented by formula (III), wherein the solid form is characterized by an x-ray powder diffraction pattern substantially in accordance with that depicted in FIG. 5. Additionally, the present invention relates to a solid form of the compound represented by formula (III), wherein the solid form is characterized by a DSC thermogram having an endothermic event at about 141° C. In certain embodiments the present invention relates to a solid form of the compound represented by formula (III), wherein the solid form is characterized by a DSC thermogram substantially in accordance with that depicted in FIG. 7.

In some embodiments the present invention relates to a solid form of the compound represented by formula (III), wherein the solid form is substantially free of solvent, for example, substantially free of water. In certain embodiments the present invention relates to a solid form of the compound represented by formula (I), wherein the solid form is anhydrous.

In some embodiments the present invention relates to a solid form of the compound represented by formula (III), wherein the solid form is substantially pure, for example substantially free of chemical impurities, or substantially free of physical impurities.

In certain embodiments the present invention relates to pharmaceutical composition comprising a solid form of the compound represented by formula (I) and a pharmaceutically acceptable carrier.

In some embodiments the present invention relates to a method of promoting myelination of central nervous system neurons in a subject suffering from a myelin-related disorder, the method comprising administering to the subject a therapeutically effective amount of the solid form of the compound represented by formula (II) or (I) or the pharmaceutical composition comprising a solid form of the compound represented by formula (II) or (I).

In certain embodiments the present invention relates to a method of promoting myelination of central nervous system neurons in a subject suffering from a myelin-related disorder, wherein the myelin-related disorder is selected from multiple sclerosis (MS), neuromyelitis optica (NMO), optic neuritis, pediatric leukodystrophies, neonatal white matter injury, age-related dementia, schizophrenia, progressive multifocal leukoencephalopathy (PML), encephalomyelitis (EPL), central pontine myelolysis (CPM), adrenoleukodystrophy, Alexander's disease, Pelizaeus Merzbacher disease (PMD), Vanishing White Matter Disease, Wallerian Degeneration, transverse myelitis, amylotrophic lateral sclerosis (ALS), Huntington's disease, Alzheimer's disease, Parkinson's disease, spinal cord injury, traumatic brain injury, post radiation injury, neurologic complications of chemotherapy, stroke, acute ischemic optic neuropathy, vitamin E deficiency, isolated vitamin E deficiency syndrome, Bassen-Kornzweig syndrome, Marchiafava-Bignami syndrome, metachromatic leukodystrophy, trigeminal neuralgia, acute disseminated encephalitis, Guillian-Barre syndrome, Charcot-Marie-Tooth disease, Bell's palsy and radiation-induced demyelination, for example, neuromyelitis optica (NMO), optic neuritis, pediatric leukodystrophies, neonatal white matter injury, age-related dementia and schizophrenia.

In certain embodiments the present invention relates to a method of promoting myelination of central nervous system neurons in a subject suffering from a myelin-related disorder, wherein the myelin-related disorder is multiple sclerosis. In some instances, the multiple sclerosis is classified as primary progressive MS (PPMS), or as relapsing and remitting MS (RRMS), or as secondary progressive MS (SPMS).

In certain embodiments the present invention relates to a method of promoting myelination of central nervous system neurons in a subject suffering from a myelin-related disorder, wherein the subject is a human, for example, a female human.

In some embodiments the present invention relates to a method of promoting myelination of central nervous system neurons in a subject suffering from a myelin-related disorder, wherein the neurons are in the brain, spinal cord, or both the brain and spinal cord.

In certain embodiments the present invention relates to a method of promoting myelination of central nervous system neurons in a subject suffering from a myelin-related disorder, wherein the pharmaceutical composition is administered intravenously, intrathecally, subcutaneously, intramuscularly, intranasally or orally. In some embodiments the method further comprises administering a therapeutically effective amount of an MS therapeutic agent, for example, glatiramer acetate, Ocrevus (ocrelizumab), Campath (Lemtrada or alemtuzumab), Gilenya, Ampyra (dalfampridine), Tysabri (natalizumab), Aubagio (teriflunomide), Rebif, Avonex, Betaseron, Plegridy, Interferon Beta-la, dimethyl fumarate, fingolimod, rituximab, Zinbryta, Ofatumymab, Nerventra (laquinimod), Masitinib, Siponimod, Ozanimod, Ponesimod, ibudilast, vatelizumab, minocycline, ibrutinib, PRN2246, Cladripine, GNBAC1, daclizumab, and MD1003 (biotin). In some embodiments the MS therapeutic agent is administered simultaneously with the solid form of the compound represented by formula (II) or (III) or the pharmaceutical composition comprising a solid form of the compound represented by formula (II) or (I). Alternatively, the MS therapeutic agent is administered prior to the administration of the solid form of the compound represented by formula (II) or (I) or the pharmaceutical composition comprising a solid form of the compound represented by formula (II) or (III). In some cases, the MS therapeutic agent is administered following the administration of the solid form of the compound represented by formula (II) or (III) or the pharmaceutical composition comprising a solid form of the compound represented by formula (II) or (III).

In certain embodiments the present invention relates to a method of promoting myelination of central nervous system neurons in a subject suffering from a myelin-related disorder, wherein the subject is administered the compound for an on-drug cycle of at least three months, or for an on-drug cycle of at least six months. In some embodiments the present invention relates to a method of promoting myelination of central nervous system neurons in a subject suffering from a myelin-related disorder, wherein the subject is administered a therapeutically effective amount of the solid form of the compound represented by formula (II) or (III) or the pharmaceutical composition comprising a solid form of the compound represented by formula (II) or (III) using the following dosing regimen:

a. on-drug cycle for at least six months;

b. off-drug cycle for at least three months;
wherein the on-drug and off-drug cycles are optionally repeated.

In some embodiments the present invention relates to a method of promoting myelination of central nervous system neurons in a subject suffering from a myelin-related disorder, wherein the compound inhibits enzyme mediate synthesis of one or more sterol intermediates in the cholesterol biosynthesis pathway, for example, wherein the compound promotes accumulation of A8,9-unsaturated sterol intermediates in the cholesterol biosynthesis pathway, or wherein the compound inhibits one or more of CYP51, sterol-14-reductase, or EBP enzyme mediated synthesis of sterol intermediates in the cholesterol biosynthesis pathway. In some embodiments the present invention relates to a method of promoting myelination of central nervous system neurons in a subject suffering from a myelin-related disorder, wherein the compound induces, promotes, and/or modulates oligodendrocyte precursor cell (OPC) differentiation, proliferation and/or maturation, for example, wherein the induction of OPC differentiation is characterized by an increase in myelin basic protein (MBP) expression. In some embodiments the present invention relates to a method of inducing endogenous oligodendrocyte precursor cell (OPC) differentiation in a subject in need thereof, the method comprising administering to the subject a therapeutically effective amount of the solid form of the compound represented by formula (II) or (III) or the pharmaceutical composition comprising a solid form of the compound represented by formula (II) or (III). In certain embodiments, the subject suffers from a myelin-related disorder, for example, multiple sclerosis. In some embodiments the subject is human.

In some embodiments the present invention relates to a method of promoting myelination of central nervous system neurons in a subject suffering from a myelin-related disorder, wherein the compound inhibits enzyme mediate synthesis of one or more sterol intermediates in the cholesterol biosynthesis pathway, further comprising administering a therapeutically effective amount of an MS therapeutic agent.

### BRIEF DESCRIPTION OF THE DRAWINGS

The foregoing will be apparent from the following more particular description of example embodiments of the invention.

FIG. 1 is an x-ray powder diffraction (XRPD) pattern of the compound of formula II.

FIG. 2 is a thermogravimetric analysis (TGA) thermogram of the of the compound of formula I.

FIG. 3 is a differential scanning calorimetry (DSC) thermogram of the compound of formula I.

FIG. 4 is a dynamic vapor sorption (DVS) pattern of the compound of formula II.

FIG. 5 is an XRPD pattern of the compound of formula III.

FIG. 6 is a TGA thermogram of the compound of formula III.

FIG. 7 is a DSC thermogram of the compound of formula III.

FIG. 8 is a DVS pattern of the compound of formula III.

FIG. 9 is an XRPD pattern of the compound of the besylate salt of cis-N-cyclohexyl-N-ethyl-3-(3-chloro-4-cyclohexylphenyl)prop-2-enylamine.

FIG. 10 is a TGA thermogram of the besylate salt of cis-N-cyclohexyl-N-ethyl-3-(3-chloro-4-cyclohexylphenyl)prop-2-enylanine.

FIG. 11 is a DSC thermogram of the besylate salt of cis-N-cyclohexyl-N-ethyl-3-(3-chloro-4-cyclohexylphenyl)prop-2-enylanine.

FIG. 12 is a DVS pattern of the besylate salt of cis-N-cyclohexyl-N-ethyl-3-(3-chloro-4-cyclohexylphenyl)prop-2-enylamine.

FIG. 13 is an XRPD pattern of the xinafoate salt of cis-N-cyclohexyl-N-ethyl-3-(3-chloro-4-cyclohexylphenyl)prop-2-enylamine.

FIG. 14 is a TGA thermogram of the xinafoate salt of cis-N-cyclohexyl-N-ethyl-3-(3-chloro-4-cyclohexylphenyl)prop-2-enylamine.

FIG. 15 is a DSC thermogram of the xinafoate salt of cis-N-cyclohexyl-N-ethyl-3-(3-chloro-4-cyclohexylphenyl)prop-2-enylamine.

FIG. 16 is a DVS pattern of the xinafoate salt of cis-N-cyclohexyl-N-ethyl-3-(3-chloro-4-cyclohexylphenyl)prop-2-enylamine.

FIG. 17 is an XRPD pattern of the edisylate salt of cis-N-cyclohexyl-N-ethyl-3-(3-chloro-4-cyclohexylphenyl)prop-2-enylamine.

FIG. 18 is a TGA thermogram of the edisylate salt of cis-N-cyclohexyl-N-ethyl-3-(3-chloro-4-cyclohexylphenyl)prop-2-enylamine.

FIG. 19 is a DSC thermogram of the edisylate salt of cis-N-cyclohexyl-N-ethyl-3-(3-chloro-4-cyclohexylphenyl)prop-2-enylamine.

FIG. 20 is a DVS pattern of the edisylate salt of cis-N-cyclohexyl-N-ethyl-3-(3-chloro-4-cyclohexylphenyl)prop-2-enylamine.

### DETAILED DESCRIPTION OF THE INVENTION

The enhancement and/or inducement of the accumulation of Δ8,9-unsaturated sterol intermediates of the cholesterol biosynthesis pathway in oligodendrocyte progenitor cells (OPCs) can induce oligodendrocyte generation. Enhancement and/or inducement of the accumulation of Δ8,9-unsaturated sterol intermediates can be provided by modulating and/or inhibiting enzymes within the cholesterol biosynthesis pathway in OPCs that inhibit Δ8,9-unsaturated sterol intermediate accumulation and/or for which the Δ8,9-unsaturated sterol intermediates are substrates as well as directly and/or indirectly administering Δ8,9-unsaturated sterol intermediates to the OPCs. Enhancement and/or inducement of the accumulation of Δ8,9-unsaturated sterol intermediates can promote OPC differentiation, survival, proliferation and/or maturation and treat disease and/or disorders in subjects where myelination is beneficial to the subject. This mechanism of promoting myelination is distinct from the primary action of immunomodulatory agents that are often used to treat myelin-related disorders.

As such, an agent that can enhance and/or induce accumulation of Δ8,9-unsaturated sterol intermediates of the cholesterol biosynthesis pathway in the OPCs can be administered to a subject and/or the OPCs at an amount effective to promote and/or induce OPC differentiation, proliferation and/or maturation as well as oligodendrocyte generation.

cis-N-cyclohexyl-N-ethyl-3-(3-chloro-4-cyclohexylphenyl)prop-2-enylamine is capable of enhancing and/or inducing accumulation of Δ8,9-unsaturated sterols through of modulation and/or inhibition of enzyme mediated conversion of Δ8,9-unsaturated sterols, such as conversion of lanosterol to FF-MAS, FF-MAS to T-MAS, zymostenol to lathosterol, T-MAS to zymosterol, zymosterol to dehydrolatho sterol and/or desmosterol to cholesterol in the cholesterol biosynthesis pathway at an amount effective to promote and/or induce oligodendrocyte precursor cell differentiation, proliferation and/or maturation (WO2018/022904A2).

Syntheses of cis-N-cyclohexyl-N-ethyl-3-(3-chloro-4-cyclohexylphenyl)prop-2-enylamine and its HCl salt has been reported (U.S. Pat. No. 5,354,781). The HCl salt of cis-N-cyclohexyl-N-ethyl-3-(3-chloro-4-cyclohexylphenyl)prop-2-enylamine has low aqueous solubility (less than 0.9 mg/ml) and shows hygroscopicity, which makes it an undesirable candidate for a pharmaceutical composition. Accordingly, there is need for crystalline forms of cis-N-cyclohexyl-N-ethyl-3-(3-chloro-4-cyclohexylphenyl)prop-2-enylamine that are thermodynamically stable and suitable for use in pharmaceutical compositions (e.g., are readily dissolvable, exhibit good flow properties and/or good chemical stability). There is a further need for crystalline forms of compound of cis-N-cyclohexyl-N-ethyl-3-(3-chloro-4-cyclohexylphenyl)prop-2-enylamine having physical properties that enable the manufacture of cis-N-cyclohexyl-N-ethyl-3-(3-chloro-4-cyclohexylphenyl)prop-2-enylamine for use in pharmaceutical compositions in high yield and high purity.

Solid state physical properties of a material affect the ease with which the material is handled during processing into a pharmaceutical product, such as a tablet or capsule formulation. The physical properties affect the types of excipients, for example, to be added to a formulation for a pharmaceutical compound. Furthermore, the solid state physical property of a pharmaceutical compound is important to its dissolution in aqueous and liquid milieus, including gastric juices, thereby having therapeutic consequences. The solid state form of a pharmaceutical compound may also affect its storage requirements. From a physicochemical perspective, the crystalline form of a pharmaceutical compound is the preferred form. Organization of the molecules in an ordered fashion to form a crystal lattice provides improved chemical stability, flowability, and other powder properties including reduced moisture sorption. Anhydrous forms are often desirable because they can be consistently made without concern for variation in weight or composition due to varying solvent or water content. All of these properties are of importance to the manufacturing, formulation, storage and overall manageability of a pharmaceutical drug product.

### Compounds

The present invention relates to pharmaceutically acceptable salts of cis-N-cyclohexyl-N-ethyl-3-(3-chloro-4-cyclohexylphenyl)prop-2-enylamine, represented by formula I, or solvates thereof:

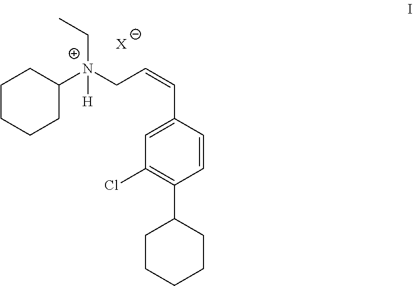

wherein:

X⁻ is selected from the group consisting of the anions of the following acids: ethanesulfonic acid (ESA), salicylic acid, adipic acid, benzenesulfonic acid (BSA), trans-cinnamic acid, ethanedisulfonic acid (EDSA), gentisic acid, glycolic acid, α-ketoglutaric acid, malic acid, malonic acid, 1,5-naphthalenedisulfonic acid (1,5-NDSA), L-tartaric acid, vanillic acid, and xinafoic acid.

In some embodiments, X⁻ is the anion of ethanesulfonic acid (esylate). In some embodiments, X⁻ is the anion of salicylic acid (salicylate). In some embodiments, X⁻ is the anion of benzenesulfonic acid (BSA), ethanedisulfonic acid (EDSA), and xinafoic acid. In some embodiments, X⁻ is the anion of adipic acid, trans-cinnamic acid, gentisic acid, glycolic acid, α-ketoglutaric acid, malic acid, malonic acid, 1,5-naphthalenedisulfonic acid (1,5-NDSA), L-tartaric acid, and vanillic acid.

The salts disclosed herein may be prepared by precipitation from organic or mixed organic solvents and may also be prepared from organic/aqueous solvents. Suitable organic solvents include acetonitrile, diethyl ether, ethyl acetate, ethanol, isopropyl alcohol, isopropyl ether, methyl ethyl ketone, methyl cyclohexane, methanol, methyl isobutyl ketone, and methyl-tert-butyl ether.

Illustrative, non-limiting examples of such preparations are given in the Example section below.

“Crystalline,” as used herein, refers to a homogeneous solid formed by a repeating, three-dimensional pattern of atoms, ions or molecules (e.g., an anhydrous molecule or a salt thereof, solvate thereof, or combination of the foregoing) having fixed distances between constituent parts. The unit cell is the simplest repeating unit in this pattern.

“Substantially free of”, as used herein, means containing no more than an insignificant amount. In some embodiments, a composition or preparation is “substantially free of” a recited element if it contains less than 5%, 4%, 3%, 2%, or 1%, by weight of the element. In some embodiments, the composition or preparation contains less than 0.9%, 0.8%, 0.7%, 0.6%, 0.5%, 0.4%, 0.3%, 0.2%, 0.1% or less of the recited element. In some embodiments, the composition or preparation contains an undetectable amount of the recited element

The crystalline forms provided herein can be identified on the basis of characteristic peaks in an x-ray powder diffraction (XRPD) analysis. XRPD is a scientific technique that measures the x-rays, neutrons or electrons scattered by a powder or microcrystalline material as a function of scattering angle. XRPD can be used to identify and characterize crystalline solids, as the diffraction pattern produced by a particular solid is typically distinctive to that solid and can be used as a “fingerprint” to identify that solid. For example, an XRPD pattern or diffractogram (e.g., a pattern or diffractogram produced by a sample, such as an unknown sample) that is substantially in accordance with a reference XRPD pattern or diffractogram can be used to determine the identity between the sample material and the reference material. Both the position and the relative intensity of the peaks in an XRPD diffractogram are indicative of the particular phase and identity of a material.

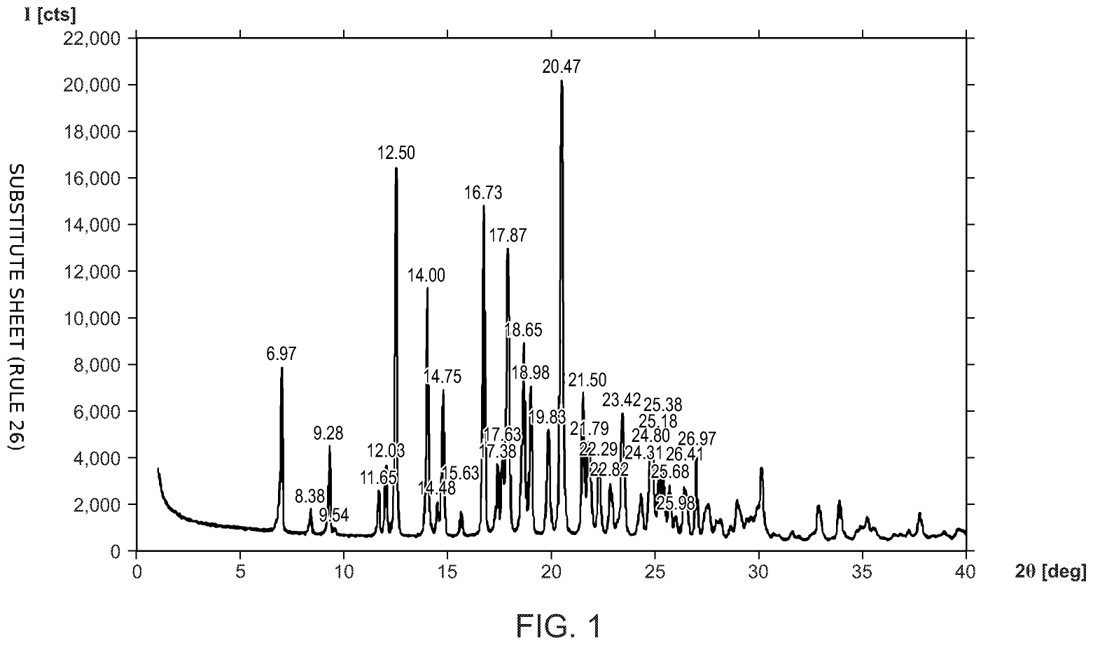

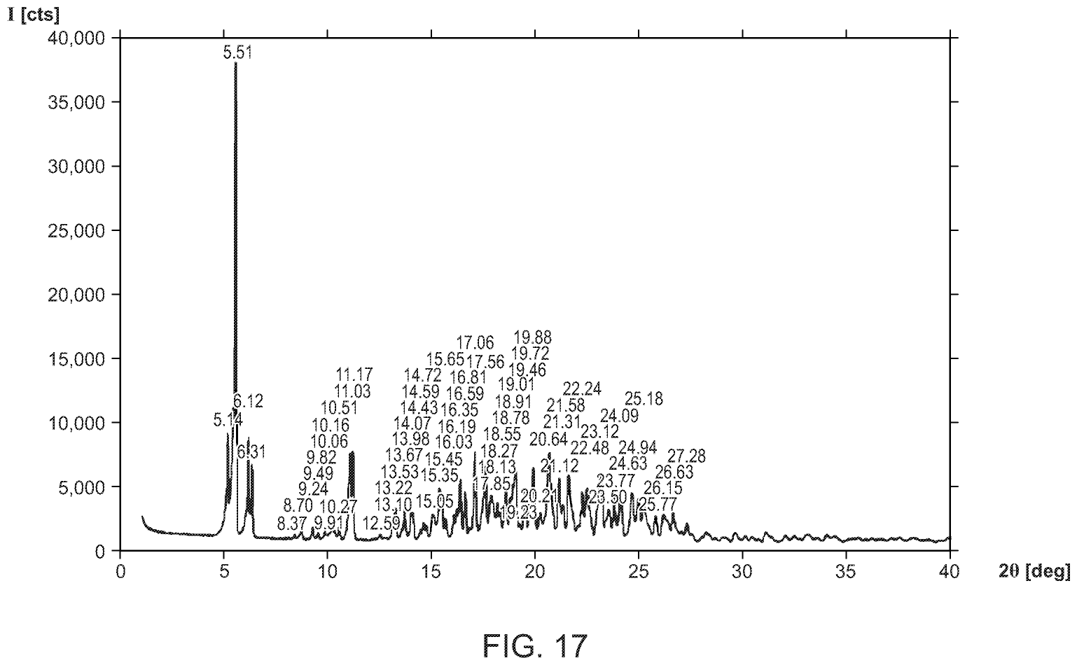

FIGS. 1, 5, 9, 13, and 17 show XRPD patterns of various salts described herein. An XRPD pattern that is “substantially in accordance” with one or more FIGS. herein showing an XRPD pattern or diffractogram is an XRPD pattern that would be considered by one skilled in the art to represent the same crystalline form of the compound of formula I as the sample of the compound of formula I that provided the XRPD pattern of one or more FIGS. provided herein. Thus, an XRPD pattern that is substantially in accordance may be identical to that of one of the FIGS. or, more likely, may be somewhat different from one or more of the FIGS. An XRPD pattern that is somewhat different from one or more of the FIGS. may not necessarily show each of the lines of the diffraction pattern presented herein and/or may show a slight change in appearance or intensity of the lines or a shift in the position of the lines. These differences typically result from differences in the conditions involved in obtaining the data or differences in the purity of the sample used to obtain the data. A person skilled in the art is capable of determining if a sample of a crystalline compound is of the same form as or a different form from a form disclosed herein by comparison of the XRPD pattern of the sample and the corresponding XRPD pattern disclosed herein.

It is to be understood that any 2θ angle specified herein means the specified value±0.2°. For example, when a described embodiment or a claim specifies a 20 of 4.4°, this is to be understood to mean 4.4°±0.2°, that is, a 2θ angle of from 4.2° to 4.6°.

The crystalline forms provided herein can also be identified on the basis of differential scanning calorimetry (DSC) and/or thermogravimetric analysis (TGA). DSC is a thermoanalytical technique in which the difference in the amount of heat required to increase the temperature of a sample is measured as a function of temperature. DSC can be used to detect physical transformations, such as phase transitions, of a sample. For example, DSC can be used to detect the temperature(s) at which a sample undergoes crystallization, melting or glass transition.

TGA is a method of thermal gravimetric analysis in which changes in physical and chemical properties of a material are measured as a function of increasing temperature (with constant heating rate) or as a function of time (with constant temperature and/or constant mass loss). TGA can provide information about physical phenomena, such as second-order phase transitions, or about chemical phenomena, such as desolvation and/or decomposition.

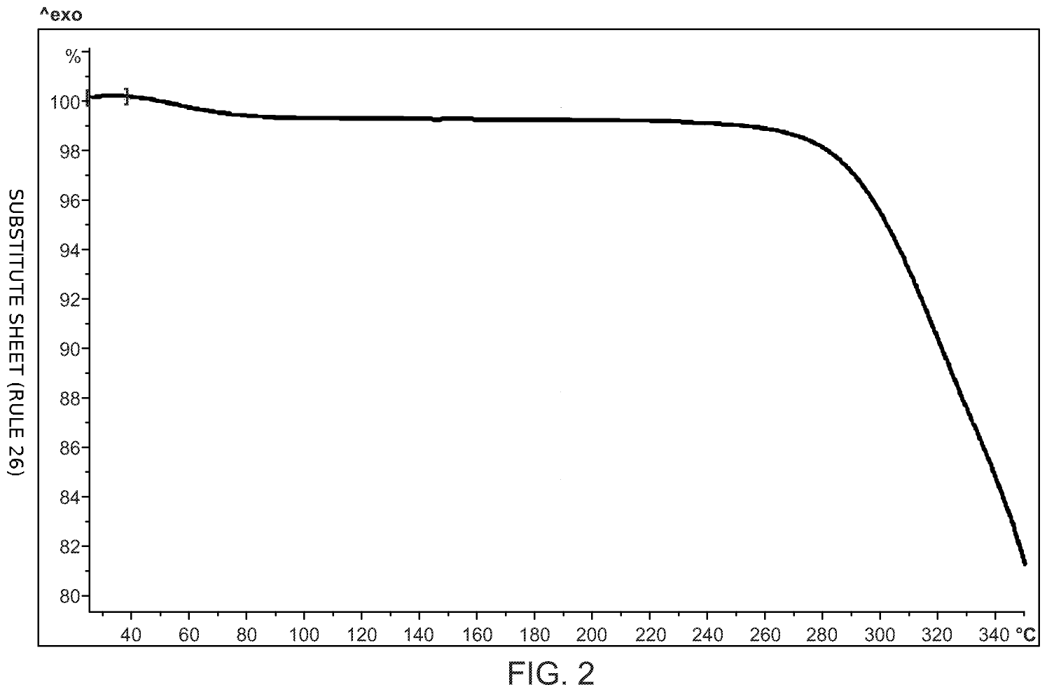

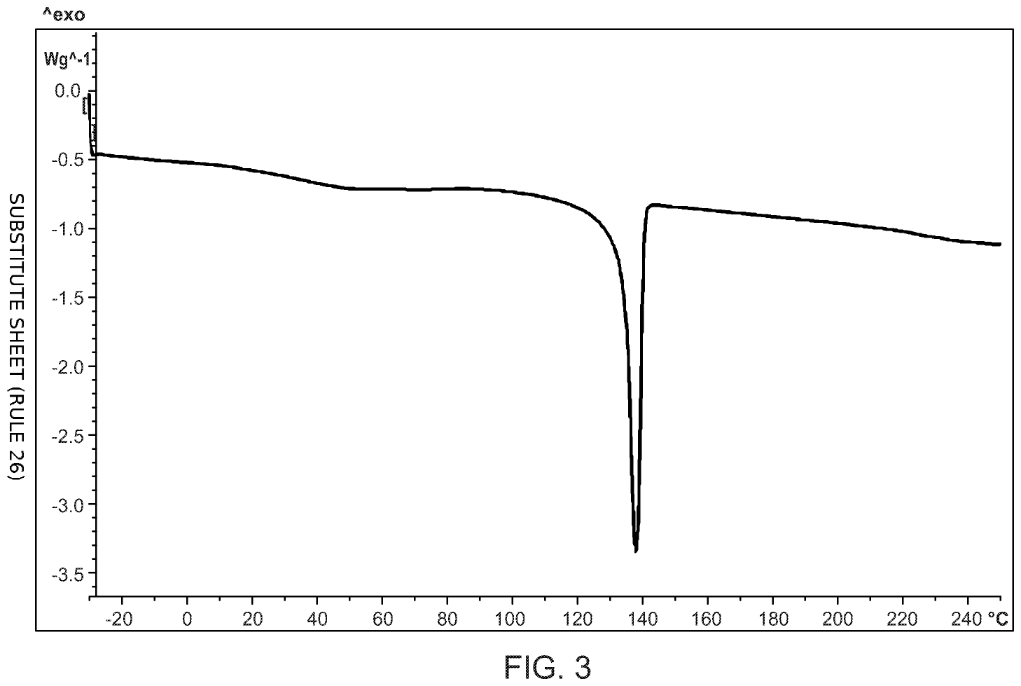

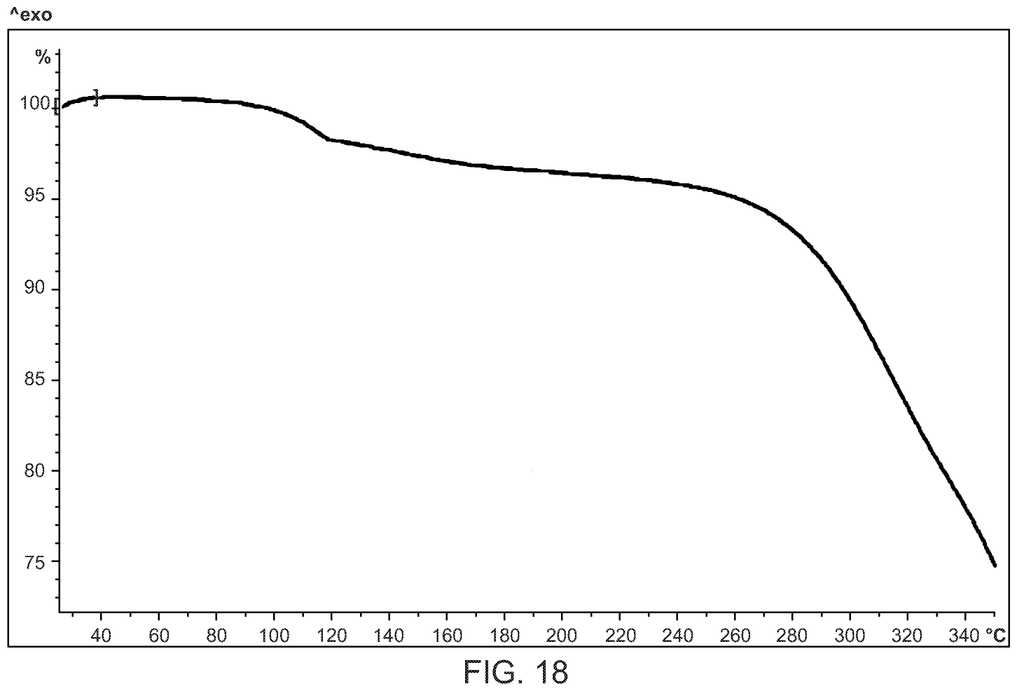

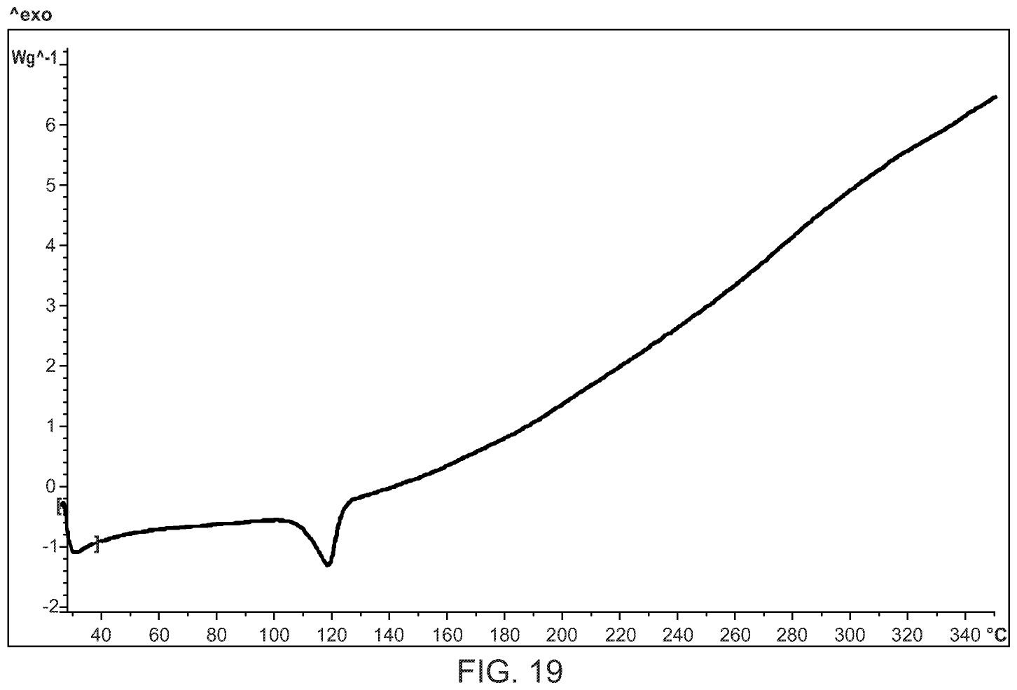

FIGS. 2, 6, 10, 14, and 18 show TGA thermograms of various crystalline forms described herein. FIGS. 3, 7, 11, 15, and 19 show DSC thermograms of various crystalline forms described herein. A DSC or TGA thermogram that is “substantially in accordance” with one or more FIGS. herein showing a DSC or TGA thermogram is a DSC or TGA thermogram that would be considered by one skilled in the art to represent the same crystalline form of the compound of formula I as the sample of the compound of formula I that provided the DSC or TGA thermogram of one or more FIGS. provided herein.

It is to be understood that any temperature associated with DSC or TGA specified herein means the specified value±5° C. or less. For example, when an embodiment or a claim specifies an endothermic peak at about 179° C., this is to be understood to mean 179° C. 5° C. or less, that is a temperature of from 174° C. to 184° C. In preferred embodiments, a DSC or TGA temperature is the specified value f 3° C., in more preferred embodiments, f 2° C.

The crystalline forms provided can be additionally characterized by dynamic vapor sorption (DVS), wherein a sample is subjected to varying conditions of humidity and temperature, and the response of the sample is measured gravimetrically. The result of a DVS analysis particularly can be a dual curve providing sample weight percent as a function of relative humidity (RH) over time, a dual curve providing sample water content as a function of RH over time, a curve providing weight percent in relation to RH, or a curve providing water content in relation to RH. Equipment useful for measuring such data is known in the art, and any such equipment can be used to measure the compounds according to the present invention. In certain embodiments, DVS analysis can be carried out by scanning at a series of specific RH values. Thus, specific crystalline forms according to the invention may be identified and described in relation to the representative graph and/or the approximate peaks obtained in DVS analysis, particularly scanning from 0% to 95% RH with a step interval of 5% or 10% RH.

Hygroscopicities of the materials described in the present disclosure are classified according to Table 1

TABLE 1
Classification of degrees of hygroscopicity for solid materials.

| Term | Definition |
|---|---|
| Low hygroscopicity | Material exhibits <0.5 wt % water |
| | uptake over a specified RH range. |
| Limited | Material exhibits <2.0 wt % water |
| hygroscopicity | uptake over a specified RH range. |
| Significant | Material exhibits ≥2.0 wt % water |
| hygroscopicity | uptake over a specified RH range. |
| Deliquescence | Spontaneous liquefaction associated |
| | with water sorption at a specified |
| | RH condition. |
| Stoichiometric | Crystalline material with a defined |
| hydrate | water content over an extended RH |
| | range. Typical stoichiometric hydrates |
| | are hemihydrates, monohydrates, |
| | sesquihydrates, dihydrates, etc. |
| Variable hydrate | Crystalline material with variable |
| | water content over an extended RH |
| | range, yet with no phase change. |

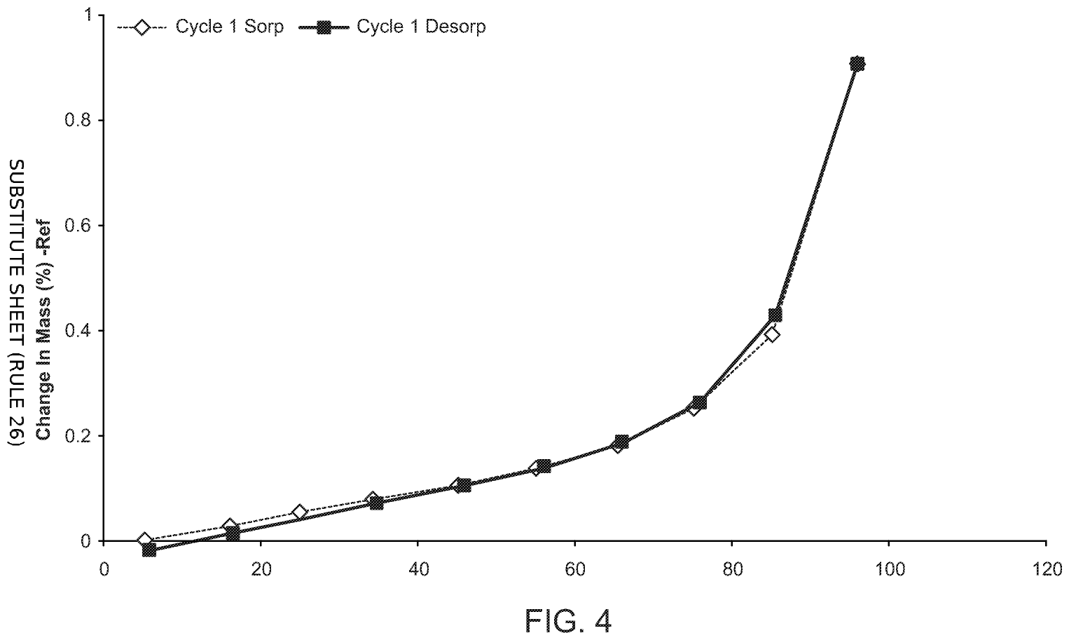

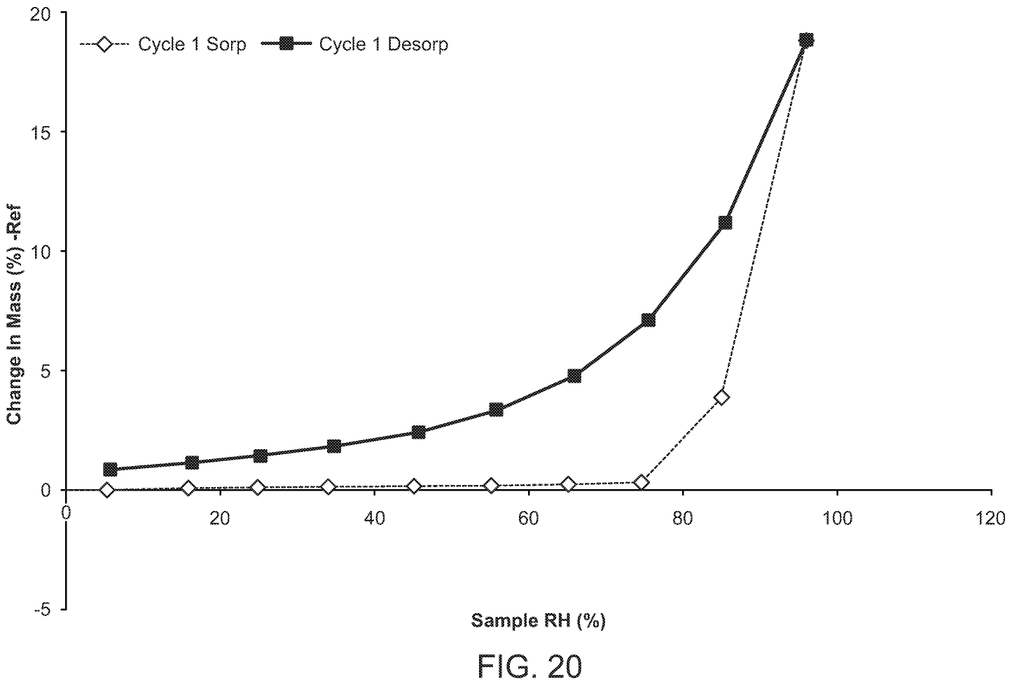

FIGS. 4, 8, 12, 16, and 20 show DVS patterns of various crystalline forms described herein. A DVS pattern that is “substantially in accordance” with one or more FIGS. herein showing a DVS patterns is a DVS pattern that would be considered by one skilled in the art to represent the same crystalline form of the compound of formula I as the sample of the compound of formula I that provided the DVS pattern of one or more FIGS. provided herein.

“Solvate,” as used herein, refers to a chemical compound formed by the interaction of a solute and one or more solvents (e.g., methanol, ethanol, water). Thus, “solvate” includes solvates containing a single type of solvent molecule and solvates containing more than one type of solvent molecule (mixed solvates or co-solvates). Typically, the one or more solvents in solvates described herein is an organic solvent or a combination of organic solvents, although water can also form solvates, called hydrates.

In some embodiments, the esylate salt of cis-N-cyclohexyl-N-ethyl-3-(3-chloro-4-cyclohexylphenyl)prop-2-enylamine, represented by formula II, is an amorphous, polymorphous, crystalline, or partially crystalline solid form. In certain embodiments, the solid form of the compound of formula II is a crystalline form. In certain embodiments, the crystalline form of the compound of formula II is characterized by at least three x-ray powder diffraction peaks at 2θ angles selected from 12.50°, 16.73°, 17.87°, and 20.47°, or at least four x-ray powder diffraction peaks at 2θ angles selected from 12.50°, 14.00°, 16.73°, 17.87°, 18.65°, and 20.47°, at least five x-ray powder diffraction peaks at 2θ angles selected from 6.97°, 12.50°, 14.00°, 14.75°, 16.73°, 17.870, 18.650, 18.980, and 20.47°.

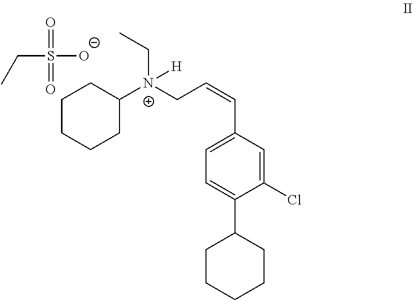

In a particular embodiment, the crystalline form of the compound of formula II is characterized by x-ray powder diffraction peaks at 2θ angles of 12.50°, 16.73°, 17.87°, and 20.47°, or by x-ray powder diffraction peaks at 2θ angles of 12.50°, 14.00°, 16.73°, 17.87°, 18.65°, and 20.47°, or by x-ray powder diffraction peaks at 2θ angles of 6.97°, 12.50°, 14.00°, 14.75°, 16.73°, 17.87°, 18.65°, 18.98°, and 20.47°. In some embodiments, the crystalline form of the compound of formula II is characterized by an x-ray powder diffraction pattern substantially in accordance with that depicted in FIG. 1.

The crystalline form of the compound of formula II may be further characterized by a differential scanning calorimetry thermogram comprising a strong endothermic peak at about 138° C. In some embodiments, the TGA thermogram and/or the DSC thermogram are substantially in accordance with those in FIG. 2 or FIG. 3.

The crystalline form of the compound of formula II can be additionally characterized by dynamic vapor sorption pattern comprising a weight gain of about 0.9 wt % in the range of 5 to 95% RH. In some embodiments the DVS pattern is substantially in accordance with the one found in FIG. 4.

In a particular embodiment, the crystalline form of the compound of formula II is in the form of a solvate, for example, a hydrate.

In some embodiments, the crystalline form of the compound of formula II is substantially pure, for example, substantially free of chemical impurities, or substantially free of physical impurities.

Also provided herein are methods for preparing crystalline forms of a compound of formula I. In certain embodiment, the invention relates to a method of preparing a crystalline form of the compound of formula II, wherein the crystalline form characterized by at least three x-ray powder diffraction peaks at 2θ angles selected from 12.50°, 16.73°, 17.87°, and 20.47°, the method comprising:
(a) dissolving cis-N-cyclohexyl-N-ethyl-3-(3-chloro-4-cyclohexylphenyl)prop-2-enylamine in anhydrous ethyl acetate;
(b) adding solid ethanesulfonic acid to the solution, thus generating a reaction mixture;
(c) allowing the reaction mixture to stir for a predetermined time at a predetermined temperature;
(d) adding heptane and diethyl ether to the reaction mixture,
(e) allowing the solvent to evaporate, forming a gel;
(e) breaking gel into smaller particles,
(f) adding methyl tert-butyl ketone to the broken gel particles and allowing the mixture to stir for a predetermined time at a predetermined; and
(g) isolating the crystalline form of compound of formula I.

In certain embodiments the reaction mixture in step (c) is stirred at a temperature in the range from about 18° C. to about 23° C. for a period of time from about 5 to about 40 hours, preferably from about 10 hours to about 30 hours, more preferably for about 24 hours.

In certain embodiments the reaction mixture in step (f) is stirred at a temperature in the range from about 18° C. to about 23° C. for a period of time from about 4 to about 10 days, preferably from about 6 days to about 8 days, more preferably for about 7 days.

In another aspect of the method of preparing the crystalline form of compound of formula II, the particles are isolated by filtration.

In some embodiments, the salicylate salt of cis-N-cyclohexyl-N-ethyl-3-(3-chloro-4-cyclohexylphenyl)prop-2-enylamine, represented by formula III, is an amorphous, polymorphous, crystalline, or partially crystalline solid form. In certain embodiments, the solid form of the compound of formula III is a crystalline form. In a certain embodiment, the crystalline form of the compound of formula III is characterized by at least three x-ray powder diffraction peaks at 2θ angles selected from 4.93°, 14.71°, 20.85°, and 25.39°, or at least four x-ray powder diffraction peaks at 2θ angles selected from 4.93°, 14.71°, 19.03°, 20.85°, 24.94°, and 25.39°, at least five x-ray powder diffraction peaks at 2θ angles selected from 4.93°, 12.79°, 14.710, 16.970, 17.590, 19.030, 20.85°, 22.08°, 24.94°, and 25.39°.

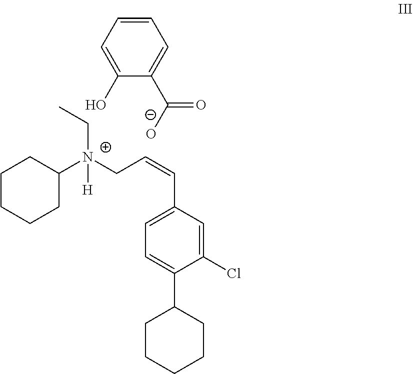 [SMILES: CC[NH+](C/C=C\c1ccc(C2CCCCC2)c(Cl)c1)C1CCCCC1 ; O=C([O-])c1ccccc1O]

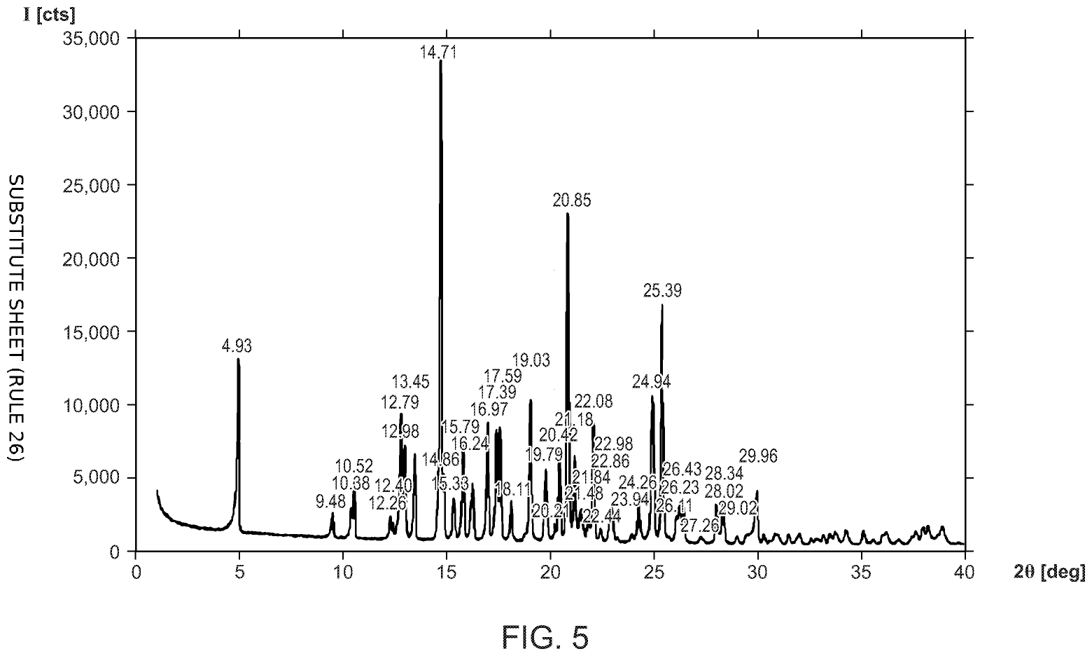

In a particular embodiment, the crystalline form of the compound of formula III is characterized by x-ray powder diffraction peaks at 2θ angles of 4.93°, 14.71°, 20.85°, and 25.39°, or by x-ray powder diffraction peaks at 2θ angles of 4.93°, 14.71°, 19.03°, 20.85°, 24.94°, and 25.39°, or by x-ray powder diffraction peaks at 2θ angles of 4.93°, 12.79°, 14.71°, 16.97°, 17.59°, 19.03°, 20.85°, 22.08°, 24.94°, and 25.39°. In some embodiments, the crystalline form of the compound of formula III is characterized by an x-ray powder diffraction pattern substantially in accordance with that depicted in FIG. 5.

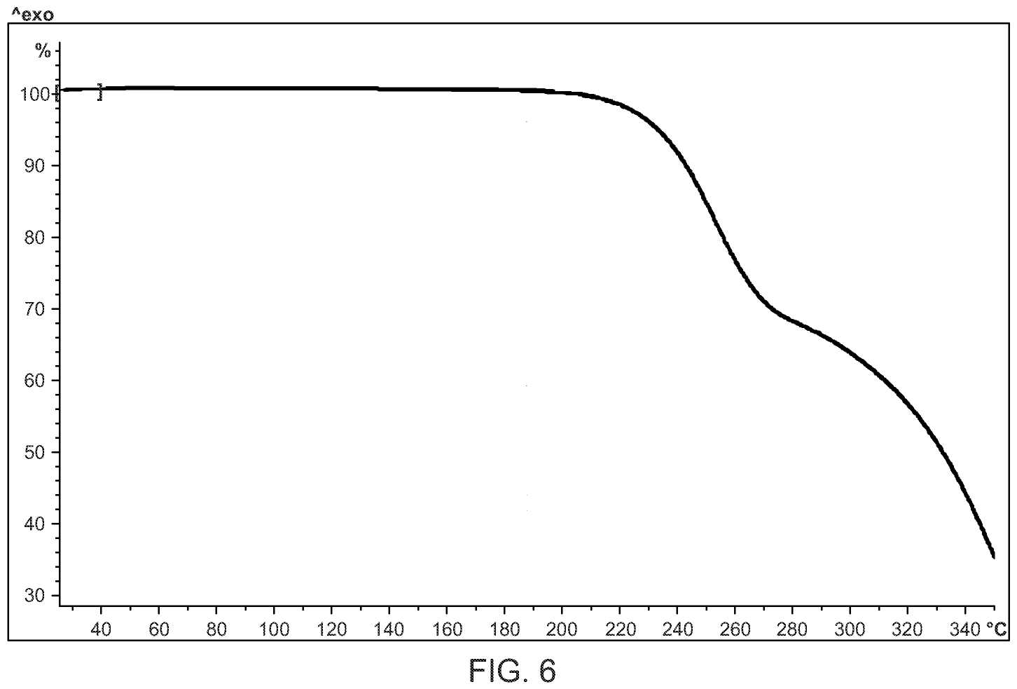

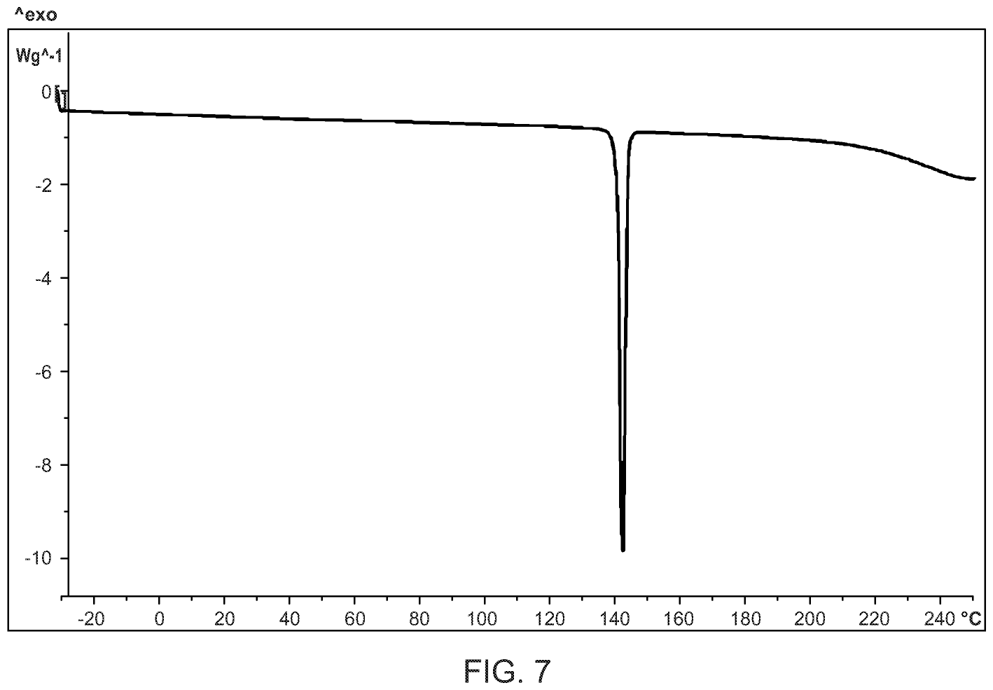

The crystalline form of the compound of formula III may be further characterized by a differential scanning calorimetry thermogram comprising a strong endothermic peak at about 141° C. In some embodiments, the TGA thermogram and/or the DSC thermogram are substantially in accordance with those in FIG. 6 or FIG. 7.

The crystalline form of the compound of formula III can be additionally characterized by dynamic vapor sorption pattern comprising a weight gain of about 0.04 wt % in the range of 5 to 95% RH. In some embodiments the DVS pattern is substantially in accordance with the one found in FIG. 4.

In some embodiments, the crystalline form of the compound of formula III is substantially pure, for example, substantially free of chemical impurities, or substantially free of physical impurities.

In a particular embodiment, the crystalline form of the compound of formula III is free of solvent. In a certain embodiment, the crystalline form of the compound of formula III is anhydrous.

Also provided herein are methods for preparing crystalline forms of a compound of formula III. In certain embodiment, the invention relates to a method of preparing a crystalline form of the compound of formula III, wherein the crystalline form characterized by at least three x-ray powder diffraction peaks at 2θ angles selected from 4.93°, 14.71°, 20.85°, and 25.39°, the method comprising:

(a) dissolving cis-N-cyclohexyl-N-ethyl-3-(3-chloro-4-cyclohexylphenyl)prop-2-enylamine in diisopropyl ether;
(b) dissolving salicylic acid in ethanol;
(c) adding the solution of the salicylic acid to the solution of cis-N-cyclohexyl-N-ethyl-3-(3-chloro-4-cyclohexylphenyl)prop-2-enylamine, thus generating a reaction mixture;
(d) adding diisopropyl ether to the reaction mixture;
(e) allowing the reaction mixture to stir for a predetermined time at a predetermined temperature;
(f) isolating the crystalline form of compound of formula III.

In certain embodiments the ratio by volume of ethanol to diisopropyl ether in step (d) is about 1, or about 1/2, or 1/5, or about 1/10, preferably about 1/4.

In certain embodiments the reaction mixture in step (e) is stirred at a temperature in the range from about 18° C. to about 23° C. for a period of time from about 1 to about 7 days, preferably from about 3 days to about 5 days, more preferably for about 4 days.

In another aspect of the method of preparing the crystalline form of compound of formula III, the particles are isolated by filtration.

Compounds of formula I were prepared by treating the free base of cis-N-cyclohexyl-N-ethyl-3-(3-chloro-4-cyclohexylphenyl)prop-2-enylamine with the corresponding acid. Detailed descriptions of the salt formation for selected compounds of formula I are presented in the Exemplification section. Salt formation experiments were conducted using a tiered screening approach and utilized various crystallization techniques and solvent systems. Solutions or suspensions of cis-N-cyclohexyl-N-ethyl-3-(3-chloro-4-cyclohexylphenyl)prop-2-enylamine were prepared and the corresponding acids were then added neat or as solutions at ambient or elevated temperature. In some instances, solutions of starting material were added to solutions or suspensions of salt formers. The resulting solutions/suspensions were stirred at ambient or elevated temperature for a given duration. If solids were not produced, further crystallization techniques were applied.

Generated solids were typically observed by polarized light microscopy and, if crystalline (i.e. display birefringence and extinction), analyzed by XRPD. The XRPD patterns were compared to each other and to available reference patterns of salt formers. If unique by XRPD, the materials were further analyzed by ¹H NMR, TGA, DSC, and DVS. Kinetic solubilities of selected materials were estimated by aliquot addition method. Equilibrium solubilities were estimated gravimetrically using supernatants from suspensions equilibrated at ambient temperature for approximately 7-10 days.

The experimental observations obtained during the treatment of the free base with a variety of acids are summarized in Table 2. In the cases when non-viscous solids were isolated, crystallinity and chemical composition of the materials was assessed using XRPD, as presented in Table 2.

TABLE 2
Formation of cis-N-cyclohexyl-N-ethyl-3-(3-chloro-
4-cyclohexylphenyl)prop-2-enylamine salts.

| Acid (a) | Observations | XRPD Results |
|---|---|---|
| Adipic acid. (1:1) | Solids formed | Crystalline material. |
| | | Adipic acid |
| Benzenesulfonic | Solids formed | Crystalline material, |
| acid (1:1) | | Besylate salt |
| trans-Cinnamic | No solids formed | N/A |
| acid (1:1) | | |
| Ethanedisulfonic | Solids formed | Crystalline material, |
| acid (2:1) | | Edisylate salt |
| Ethanedisulfonic | Gel formed | N/A |
| acid (1:1) | | |
| Ethanesulfonic | Solids formed | Crystalline material, |
| acid (1:1) | | Esylate salt |
| Gentisic acid (1:1) | Gel or viscous solids | N/A |
| | formed | |
| Glycolic acid (1:1) | No solids formed | N/A |
| α-Ketoglutaric acid | No solids formed | N/A |
| Malic acid (1:1) | Gel or viscous material | N/A |
| | formed | |
| Malic acid (2:1) | Viscous solids formed | N/A |
| Malonic acid (1:1) | Gel or viscous material | N/A |
| | formed | |
| Malonic acid (2:1) | Viscous solids formed | N/A |
| 1,5- | Solids formed | Amorphous material |
| Naphthalenedisulfonic | | |
| acid (1:1) | | |
| L-Tartaric acid (1:1) | Viscous solids formed | N/A |
| L-Tartaric acid (2:1) | No solids formed | N/A |
| Salicylic acid (1.1) | Solids formed | Crystalline |
| | | material, |
| | | Salicylate salt |
| Vanillic acid. (1:1) | Solids formed | Crystalline |
| | | material, |
| | | Vanillic acid |
| Xinafoic acid (1:1) | Solids formed | Crystalline |
| | | material, |
| | | Xinafoate salt |

(a) Approximate free base: acid molar ratio.

The physicochemical properties of the obtained crystalline salts are summarized below.

Esylate salt is an unsolvated and possibly anhydrous crystalline salt of cis-N-cyclohexyl-N-ethyl-3-(3-chloro-4-cyclohexylphenyl)prop-2-enylamine with a 1:1 stoichiometry. It shows limited hygroscopicity upon increase of RH, but undergoes a complete desorption without hysteresis or form change. Melting temperature of the salt is 138° C. (DSC). Decomposition onset at 268° C. was determined by TGA. The salt provides a significant improvement over the HCl salt with regards to aqueous solubility, and has a kinetic estimate of 30 mg/mL (versus less than 0.9 mg/mL for the HCl salt).

Salicylate salt is an unsolvated and anhydrous crystalline salt of cis-N-cyclohexyl-N-ethyl-3-(3-chloro-4-cyclohexylphenyl)prop-2-enylamine with a 1:1 stoichiometry. Melting temperature of the salt is 141° C. (DSC). Decomposition onset at 212° C. was determined by TGA. Its aqueous kinetic solubility is less than 1 mg/mL, and it is thermally stable and is non-hygroscopic over the entire RH range.

Besylate slat is a crystalline salt of cis-N-cyclohexyl-N-ethyl-3-(3-chloro-4-cyclohexylphenyl)prop-2-enylamine with a 1:1 stoichiometry. It is unsolvated and likely anhydrous. Melting temperature of the salt is 108° C. (DSC). Decomposition onset at 274° C. was determined by TGA. It displays limited hygroscopicity upon increase of RH and undergoes complete desorption without form change and with almost no hysteresis. The salt has a lower melting point than the other crystalline salts and does not show improvement with regards to aqueous kinetic solubility (<1 mg/mL).

Xinafoate salt is a crystalline salt of cis-N-cyclohexyl-N-ethyl-3-(3-chloro-4-cyclohexylphenyl)prop-2-enylamine with a 1:1 stoichiometry, is unsolvated and is possibly a variable hydrate or anhydrous. The salt is non-hygroscopic below 65% RH, has limited hygroscopicity above 65% RH, but undergoes a complete desorption. Melting temperature of the salt is 137° C. (DSC). Decomposition onset at 146° C. was determined by TGA. Suspected solid-solid transition or rapid melt/recrystallization was detected around 41-42° C. The aqueous kinetic solubility is less than 1 mg/mL. A form change is suggested after moisture sorption-desorption cycles (XRPD).

Edisylate salt is a crystalline salt with a 2:1 mol:mol ratio of cis-N-cyclohexyl-N-ethyl-3-(3-chloro-4-cyclohexylphenyl)prop-2-enylamine and ethanedisulfonic acid. It is likely hydrated and/or solvated, or alternatively, could be a mixed hydrate/solvate. It is non-hygroscopic below 75% RH; however, above 75% RH it displays significant hygroscopicity and deliquesces. A form change to a highly disordered material is observed after moisture sorption-desorption cycles. Melting temperature of the salt is 119° C. (DSC). Decomposition onset at 260° C. was determined by TGA.

The experimental data for the compounds of formula I presented above show that out of 15 acids treated with cis-N-cyclohexyl-N-ethyl-3-(3-chloro-4-cyclohexylphenyl)prop-2-enylamine only 5 provided crystalline salts under a variety of examined crystallization conditions. Furthermore, of the five crystalline salts two, the esylate and salicylate, show unexpected favorable physicochemical characteristics, such as high solubility of the esylate and thermal stability and lack of hygroscopicity of the salicylate. These observations demonstrate that the selection of acid and crystallization conditions in order to get a crystalline salt of cis-N-cyclohexyl-N-ethyl-3-(3-chloro-4-cyclohexylphenyl)prop-2-enylamine with favorable physicochemical characteristics would not be obvious to a person of ordinary skill in the art

### Pharmaceutical Compositions

In some embodiments, the invention relates to a composition, comprising particles of the crystalline form of the compound of formula II, wherein the crystalline form is characterized by x-ray powder diffraction peaks at 2θ angles of 12.50°, 16.73°, 17.87°, and 20.47. In certain embodiments, the invention relates to the compositions and pharmaceutical compositions as described herein, wherein the crystalline form of the compound of formula II is characterized by at least three x-ray powder diffraction peaks at 2θ angles selected from 12.50°, 16.73°, 17.87°, and 20.47°, or at least four x-ray powder diffraction peaks at 2θ angles selected from 12.50°, 14.00°, 16.73°, 17.87°, 18.65°, and 20.47°, at least five x-ray powder diffraction peaks at 28 angles selected from 6.97°, 12.50°, 14.00°, 14.75°, 16.73°, 17.87°, 18.65°, 18.98°, and 20.47°.

In certain embodiments, the invention relates to compositions and pharmaceutical compositions described herein, wherein the crystalline form of the compound of formula II is characterized by x-ray powder diffraction peaks at 2θ angles of 12.50°, 16.73°, 17.87°, and 20.47°, or by x-ray powder diffraction peaks at 2θ angles of 12.50°, 14.00°, 16.73°, 17.87°, 18.65°, and 20.47°, or by x-ray powder diffraction peaks at 2θ angles of 6.97°, 12.50°, 14.00°, 14.75°, 16.73°, 17.87°, 18.65°, 18.98°, and 20.47°.

In certain embodiments, the invention relates to compositions and pharmaceutical compositions described herein, wherein the crystalline form is characterized by an x-ray powder diffraction pattern substantially in accordance with that depicted in FIG. 1.

In another embodiment, the invention relates to a composition, comprising particles of the crystalline form of the compound of formula III, wherein the crystalline form is characterized by x-ray powder diffraction peaks at 2θ angles of 4.93°, 14.71°, 20.85°, and 25.39°. In certain embodiments, the invention relates to the compositions and pharmaceutical compositions as described herein, wherein the crystalline form of the compound of formula III is characterized by at least three x-ray powder diffraction peaks at 2θ angles selected from 4.93°, 14.71°, 20.85°, and 25.39°, or at least four x-ray powder diffraction peaks at 2θ angles selected from 4.93°, 14.71°, 19.03°, 20.85°, 24.94°, and 25.39°, at least five x-ray powder diffraction peaks at 20 angles selected from 4.93°, 12.79°, 14.71°, 16.97°, 17.59°, 19.03°, 20.85°, 22.08°, 24.94°, and 25.39°.

In certain embodiments, the invention relates to compositions and pharmaceutical compositions described herein, wherein the crystalline form of the compound of formula III is characterized by x-ray powder diffraction peaks at 2θ angles of 4.93°, 14.71°, 20.85°, and 25.39°, or by x-ray powder diffraction peaks at 2θ angles of 4.93°, 14.71°, 19.03°, 20.85°, 24.94°, and 25.39°, or by x-ray powder diffraction peaks at 2θ angles of 4.93°, 12.79°, 14.71°, 16.97°, 17.59°, 19.03°, 20.85°, 22.08°, 24.94°, and 25.39°.

In certain embodiments, the invention relates to compositions and pharmaceutical compositions described herein, wherein the crystalline form is characterized by an x-ray powder diffraction pattern substantially in accordance with that depicted in FIG. 5.

The term “pharmaceutically acceptable carrier” means a non-toxic solvent, dispersant, excipient, adjuvant or other material which is mixed with the active ingredient in order to permit formation of a pharmaceutical composition, i.e., a dosage form capable of being administered to a subject. A “pharmaceutically acceptable carrier” should not destroy the activity of the compound with which it is formulated. Pharmaceutically acceptable carriers are well known in the art.

Pharmaceutically acceptable carriers, adjuvants or vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Pharmaceutical compositions of the invention may be administered orally, parenterally (including subcutaneous, intramuscular, intravenous and intradermal), by inhalation spray, topically, rectally, nasally, buccally, vaginally or via an implanted reservoir. In some embodiments, provided pharmaceutical compositions are administrable intravenously and/or intraperitoneally.

The pharmaceutical compositions of the invention include ocular, oral, nasal, transdermal, topical with or without occlusion, intravenous (both bolus and infusion), and injection (intraperitoneally, subcutaneously, intramuscularly, intratumorally, or parenterally). The composition may be in a dosage unit such as a tablet, pill, capsule, powder, granule, liposome, ion exchange resin, sterile ocular solution, or ocular delivery device (such as a contact lens and the like facilitating immediate release, timed release, or sustained release), parenteral solution or suspension, metered aerosol or liquid spray, drop, ampoule, auto-injector device, or suppository; for administration ocularly, orally, intranasally, sublingually, parenterally, or rectally, or by inhalation or insufflation.

Compositions of the invention suitable for oral administration include solid forms such as pills, tablets, caplets, capsules (each including immediate release, timed release, and sustained release formulations), granules and powders; and, liquid forms such as solutions, syrups, elixirs, emulsions, and suspensions. Forms useful for ocular administration include sterile solutions or ocular delivery devices. Forms useful for parenteral administration include sterile solutions, emulsions, and suspensions.

The compositions of the invention may be administered in a form suitable for once-weekly or once-monthly administration. For example, an insoluble salt of the active compound may be adapted to provide a depot preparation for intramuscular injection (e.g., a decanoate salt) or to provide a solution for ophthalmic administration.

The dosage form containing the composition of the invention contains an effective amount of the active ingredient necessary to provide a therapeutic effect. The composition may contain from about 5,000 mg to about 0.5 mg (preferably, from about 1,000 mg to about 0.5 mg) of a compound of the invention or salt form thereof and may be constituted into any form suitable for the selected mode of administration. The composition may be administered about 1 to about 5 times per day. Daily administration or post-periodic dosing may be employed.

For oral administration, the composition is preferably in the form of a tablet or capsule containing, e.g., 500 to 0.5 milligrams of the active compound. Dosages will vary depending on factors associated with the particular patient being treated (e.g., age, weight, diet, and time of administration), the severity of the condition being treated, the compound being employed, the mode of administration, and the strength of the preparation.

The oral composition is preferably formulated as a homogeneous composition, wherein the active ingredient is dispersed evenly throughout the mixture, which may be readily subdivided into dosage units containing equal amounts of a compound of the invention. Preferably, the compositions are prepared by mixing a compound of the invention (or pharmaceutically acceptable salt thereof) with one or more optionally present pharmaceutical carriers (such as a starch, sugar, diluent, granulating agent, lubricant, glidant, binding agent, and disintegrating agent), one or more optionally present inert pharmaceutical excipients (such as water, glycols, oils, alcohols, flavoring agents, preservatives, coloring agents, and syrup), one or more optionally present conventional tableting ingredients (such as corn starch, lactose, sucrose, sorbitol, talc, stearic acid, magnesium stearate, dicalcium phosphate, and any of a variety of gums), and an optional diluent (such as water).

Binder agents include starch, gelatin, natural sugars (e.g., glucose and beta-lactose), corn sweeteners and natural and synthetic gums (e.g., acacia and tragacanth). Disintegrating agents include starch, methyl cellulose, agar, and bentonite.

Tablets and capsules represent an advantageous oral dosage unit form. Tablets may be sugarcoated or filmcoated using standard techniques. Tablets may also be coated or otherwise compounded to provide a prolonged, control-release therapeutic effect. The dosage form may comprise an inner dosage and an outer dosage component, wherein the outer component is in the form of an envelope over the inner component. The two components may further be separated by a layer which resists disintegration in the stomach (such as an enteric layer) and permits the inner component to pass intact into the duodenum or a layer which delays or sustains release. A variety of enteric and non-enteric layer or coating materials (such as polymeric acids, shellacs, acetyl alcohol, and cellulose acetate or combinations thereof) may be used.

Compounds of the invention may also be administered via a slow release composition; wherein the composition includes a compound of the invention and a biodegradable slow release carrier (e.g., a polymeric carrier) or a pharmaceutically acceptable non-biodegradable slow release carrier (e.g., an ion exchange carrier).

Biodegradable and non-biodegradable slow release carriers are well known in the art. Biodegradable carriers are used to form particles or matrices which retain an active agent(s) and which slowly degrade/dissolve in a suitable environment (e.g., aqueous, acidic, basic and the like) to release the agent. Such particles degrade/dissolve in body fluids to release the active compound(s) therein. The particles are preferably nanoparticles (e.g., in the range of about 1 to 500 nm in diameter, preferably about 50-200 nm in diameter, and most preferably about 100 nm in diameter). In a process for preparing a slow release composition, a slow release carrier and a compound of the invention are first dissolved or dispersed in an organic solvent. The resulting mixture is added into an aqueous solution containing an optional surface-active agent(s) to produce an emulsion. The organic solvent is then evaporated from the emulsion to provide a colloidal suspension of particles containing the slow release carrier and the compound of the invention.

The crystalline forms of the compounds of formulas II and III may be incorporated for administration orally or by injection in a liquid form such as aqueous solutions, suitably flavored syrups, aqueous or oil suspensions, flavored emulsions with edible oils such as cottonseed oil, sesame oil, coconut oil or peanut oil and the like, or in elixirs or similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone, and gelatin. The liquid forms in suitably flavored suspending or dispersing agents may also include synthetic and natural gums. For parenteral administration, sterile suspensions and solutions are desired. Isotonic preparations, which generally contain suitable preservatives, are employed when intravenous administration is desired.

The compounds may be administered parenterally via injection. A parenteral formulation may consist of the active ingredient dissolved in or mixed with an appropriate inert liquid carrier. Acceptable liquid carriers usually comprise aqueous solvents and other optional ingredients for aiding solubility or preservation. Such aqueous solvents include sterile water, Ringer's solution, or an isotonic aqueous saline solution. Other optional ingredients include vegetable oils (such as peanut oil, cottonseed oil, and sesame oil), and organic solvents (such as solketal, glycerol, and formyl). A sterile, non-volatile oil may be employed as a solvent or suspending agent. The parenteral formulation is prepared by dissolving or suspending the active ingredient in the liquid carrier whereby the final dosage unit contains from 0.005 to 10% by weight of the active ingredient. Other additives include preservatives, isotonizers, solubilizers, stabilizers, and pain-soothing agents. Injectable suspensions may also be prepared, in which case appropriate liquid carriers, suspending agents and the like may be employed.

Compounds of the invention may be administered intranasally using a suitable intranasal vehicle.

Compounds of the invention may also be administered topically using a suitable topical transdermal vehicle or a transdermal patch.

For ocular administration, the composition is preferably in the form of an ophthalmic composition. The ophthalmic compositions are preferably formulated as eye-drop formulations and filled in appropriate containers to facilitate administration to the eye, for example a dropper fitted with a suitable pipette. Preferably, the compositions are sterile and aqueous based, using purified water. In addition to the compound of the invention, an ophthalmic composition may contain one or more of: a) a surfactant such as a polyoxyethylene fatty acid ester; b) a thickening agents such as cellulose, cellulose derivatives, carboxyvinyl polymers, polyvinyl polymers, and polyvinylpyrrolidones, typically at a concentration in the range of about 0.05 to about 5.0% (wt/vol); c) (as an alternative to or in addition to storing the composition in a container containing nitrogen and optionally including a free oxygen absorber such as Fe), an anti-oxidant such as butylated hydroxyanisol, ascorbic acid, sodium thiosulfate, or butylated hydroxytoluene at a concentration of about 0.00005 to about 0.1% (wt/vol); d) ethanol at a concentration of about 0.01 to 0.5% (wt/vol); and e) other excipients such as an isotonic agent, buffer, preservative, and/or pH-controlling agent. The pH of the ophthalmic composition is desirably within the range of 4 to 8.

### Methods of Treatment

The present invention also provides a method for promoting myelination of central nervous system neurons in a subject suffering from a myelin-related disorder, the method comprising administering to the subject a therapeutically effective amount of the crystalline form of the compound of formula II or III, or a pharmaceutical composition comprising the same. Such myelin-related disorders include, but are not limited to, multiple sclerosis (MS), neuromyelitis optica (NMO), optic neuritis, pediatric leukodystrophies, neonatal white matter injury, age-related dementia, schizophrenia, progressive multifocal leukoencephalopathy (PML), encephalomyelitis (EPL), central pontine myelolysis (CPM), adrenoleukodystrophy, Alexander's disease, Pelizaeus Merzbacher disease (PMD), Vanishing White Matter Disease, Wallerian Degeneration, transverse myelitis, amylotrophic lateral sclerosis (ALS), Huntington's disease, Alzheimer's disease, Parkinson's disease, spinal cord injury, traumatic brain injury, post radiation injury, neurologic complications of chemotherapy, stroke, acute ischemic optic neuropathy, vitamin E deficiency, isolated vitamin E deficiency syndrome, Bassen-Kornzweig syndrome, Marchiafava-Bignami syndrome, metachromatic leukodystrophy, trigeminal neuralgia, acute disseminated encephalitis, Guillian-Barre syndrome, Charcot-Marie-Tooth disease, Bell's palsy and radiation-induced demyelination.

The crystalline form of the compound of formula II or III can be administered alone or in combination with another agent to a subject suffering from a myelin-related disorder to promote myelination of neurons (e.g., neuronal axons). A myelin-related disorder can include any disease, condition (e.g., those occurring from traumatic spinal cord injury and cerebral infarction), or disorder resulting in abnormalities of the myelin sheath. Abnormalities can be caused by loss of myelin referred to as demyelination, dysfunctional myelin referred to as dysmyelination or failure to form enough myelin referred to as hypomyelination. A myelin related disorder as used herein can arise from a genetic disorder or from a variety of neurotoxic insults.

An “on-drug cycle” is the period of time (e.g., number of days or weeks) deemed appropriate by a skilled medical professional that the drug is being administered to the subject, and will vary depending on the nature of the disease, the dose of the drug being administered, the health of the patient, the intended result, and the like. An “off-drug cycle” is the period of time between on-drug cycles. By way of example, a cycle of treatment regimen for treating multiple sclerosis can be on-drug cycle for at least six months, followed by an off drug cycle for at least three months, wherein the on-drug and off-drug cycles are optionally repeated. As will be appreciated by those of skill in the art, a cycle having any combination of the number of “on” and “off” drug days can be designed as deemed appropriate by a skilled medical professional.

Administration methods include administering an effective amount (i.e., an effective amount) of a compound or composition of the invention at different times during the course of therapy or concurrently in a combination form. The methods of the invention include all known therapeutic treatment regimens. In certain embodiments, the compound or pharmaceutical composition is administered intravenously, intrathecally, subcutaneously, intramuscularly, intranasally, or orally.

As used herein, the term “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

As used herein, the term “treating” or “treatment” refers to obtaining desired pharmacological and/or physiological effect. The effect can be prophylactic or therapeutic, which includes achieving, partially or substantially, one or more of the following results: partially or totally reducing the extent of the disease, disorder or syndrome; ameliorating or improving a clinical symptom or indicator associated with the disorder; delaying, inhibiting or decreasing the likelihood of the progression of the disease, disorder or syndrome; or partially or totally delaying, inhibiting or reducing the likelihood of the onset or development of disease, disorder or syndrome.

“Prodrug” means a pharmaceutically acceptable form of an effective derivative of a compound (or a salt thereof) of the invention, wherein the prodrug may be: 1) a relatively active precursor which converts in vivo to a compound of the invention; 2) a relatively inactive precursor which converts in vivo to a compound of the invention; or 3) a relatively less active component of the compound that contributes to therapeutic activity after becoming available in vivo (i.e., as a metabolite). See “Design of Prodrugs”, ed. H. Bundgaard, Elsevier, 1985.

“Metabolite” means a pharmaceutically acceptable form of a metabolic derivative of a compound (or a salt thereof) of the invention, wherein the derivative is an active compound that contributes to therapeutic activity after becoming available in vivo.

“Effective amount” means that amount of active compound agent that elicits the desired biological response in a subject. Such response includes alleviation of the symptoms of the disease or disorder being treated. The effective amount of a compound of the invention in such a therapeutic method is from about 0.01 mg/kg/day to about 1000 mg/kg/day, from about 0.1 mg/kg/day to about 100 mg/kg/day, from about 0.5 mg/kg/day to about 50 mg/kg/day, or from about 1 mg/kg/day to 10 mg/kg/day.

“Pharmaceutically acceptable carrier” means compounds and compositions that are of sufficient purity and quality for use in the formulation of a composition of the invention and that, when appropriately administered to an animal or human, do not produce an adverse reaction.

An embodiment of the invention includes inducing endogenous oligodendrocyte precursor cell (OPC) differentiation in a subject in need thereof, the method comprising administering to the subject a therapeutically effective amount of the crystalline form of the compound of formula II or III or a pharmaceutical composition comprising the same. In certain embodiments, the subject is suffering from a myelin-related disorder, such as multiple sclerosis. In certain embodiments, the subject is human.

In certain embodiments, the methods of inducing endogenous OPC differentiation further comprise administering a therapeutically effective amount of an MS therapeutic agent.

“Demyelination” as used herein, refers to the act of demyelinating, or the loss of the myelin sheath insulating the nerves, and is the hallmark of myelin-related disorders.

Myelin related disorders include, but are not limited to, multiple sclerosis (MS), neuromyelitis optica (NMO), optic neuritis, pediatric leukodystrophies, neonatal white matter injury, age-related dementia, schizophrenia, progressive multifocal leukoencephalopathy (PML), encephalomyelitis (EPL), central pontine myelolysis (CPM), adrenoleukodystrophy, Alexander's disease, Pelizaeus Merzbacher disease (PMD), Vanishing White Matter Disease, Wallerian Degeneration, transverse myelitis, amylotrophic lateral sclerosis (ALS), Huntington's disease, Alzheimer's disease, Parkinson's disease, spinal cord injury, traumatic brain injury, post radiation injury, neurologic complications of chemotherapy, stroke, acute ischemic optic neuropathy, vitamin E deficiency, isolated vitamin E deficiency syndrome, Bassen-Kornzweig syndrome, Marchiafava-Bignami syndrome, metachromatic leukodystrophy, trigeminal neuralgia, acute disseminated encephalitis, Guillian-Barre syndrome, Charcot-Marie-Tooth disease, Bell's palsy and radiation-induced demyelination.

Both acquired and inherited myelin disorders share a poor prognosis leading to major disability. Thus, some embodiments of the present invention can include methods for the treatment of myelin-related disorders in a subject.

In certain embodiments, the myelin-related disorder is selected from neuromyelitis optica (NMO), optic neuritis, pediatric leukodystrophies, neonatal white matter injury, age-related dementia and schizophrenia.

In certain embodiments, the myelin-related disorder is multiple sclerosis.

Multiple sclerosis (MS) is one of the most common neurological disorders frequently leading to permanent disability in young adults. The clinical course is unpredictable and highly variable. The most common disease course for MS is relapsing-remitting MS (RRMS) characterized by episodes of acute exacerbations, followed by partial or complete recovery of the deficits. About 85% of people diagnosed with MS are initially diagnosed with RRMS. RRMS typically begins in the second or third decade of life and after a medium time to conversion of around 19 years approximately 70% of the patients subsequently develop secondary progressive MS (SPMS). Secondary progressive multiple sclerosis is a form of MS that typically follows relapsing-remitting multiple sclerosis. The rate of conversion to SPMS is approximately 2-3% per year. Secondary progression is usually defined as a period of clinical worsening and steady accumulation of disability, which is independent of relapses and sustained for at least six months. When an attack does occur, recovery is usually slow and, in many cases, incomplete. Existing symptoms can get worse and physical mobility becomes increasingly difficult. The time of conversion is sometimes difficult to pinpoint as it slowly builds up and remains unnoticed by the patient and the clinician for some time. Another challenge is to distinguish the chronic progression from residual symptoms that remain after patients have experienced acute relapses. Another disease course for MS is primary progressive MS (PPMS) characterized by worsening neurologic function (accumulation of disability) from the onset of symptoms, without early relapses or remissions. Approximately 15% of people with MS are diagnosed with PPMS. Primary progressive multiple sclerosis is identified by steadily worsening neurologic functions from the onset of symptoms without distinct relapses (attacks or exacerbations) or remission. The rate of progression may vary with occasional plateaus and temporary minor improvements, but declining neurologic progression is continuous.

In certain embodiments, the MS is classified as primary progressive MS (PPMS).

In certain embodiments, the MS is classified as relapsing and remitting MS (RRMS).

In certain embodiments, the MS is classified as secondary progressive MS (SPMS).

In one embodiment, the subject suffering from multiple sclerosis has an EDSS score from about 1 to about 9.5. In a particular aspect, the subject suffering from multiple sclerosis has an EDSS score from about 2 to about 8.5. In another aspect, the subject suffering from multiple sclerosis has an EDSS score from about 2.5 to about 8.0. In a further aspect, the subject suffering from multiple sclerosis has an EDSS score from about 3.0 to about 7.5, such as from about 3.0 to about 7, from about 3.0 to about 6.5, from about 3.5 to about 6.5 or from about 4.0 to about 6.5.

In another embodiment, the subject suffering from multiple sclerosis has an EDSS score of at least 1.5. In one aspect, the subject suffering from multiple sclerosis has an EDSS score of at least 2.0. In another aspect, the subject suffering from multiple sclerosis has an EDSS score of at least 2.5. In yet another aspect, the subject suffering from multiple sclerosis has an EDSS score of at least 3.0. In another aspect, the subject suffering from multiple sclerosis has an EDSS score of at least 3.5. In a further aspect, the subject suffering from multiple sclerosis has an EDSS score of at least 4.0. In another aspect, the subject suffering from multiple sclerosis has an EDSS score of at least 4.5. In another aspect, the subject suffering from multiple sclerosis has an EDSS score of at least 5.0. In another aspect, the subject suffering from multiple sclerosis has an EDSS score of at least 5.5. In another aspect, the subject suffering from multiple sclerosis has an EDSS score of at least 6.0. In another aspect, the subject suffering from multiple sclerosis has an EDSS score of at least 6.5. In another aspect, the subject suffering from multiple sclerosis has an EDSS score of at least 7.0. In another aspect, the subject suffering from multiple sclerosis has an EDSS score of at least 7.5. In another aspect, the subject suffering from multiple sclerosis has an EDSS score of at least 8.0. In another aspect, the subject suffering from multiple sclerosis has an EDSS score of at least 8.5. In another aspect, the subject suffering from multiple sclerosis has an EDSS score of at least 9.0.

In another embodiment the subject suffering from multiples sclerosis has an EDSS score of 1.0, 1.5, 2.0, 2.5, 3.0. 3.5, 4.0, 4.5, 5.0, 5.5, 6.0, 6.5, 7.0, 7.5, 8.0, 8.5, 9.0 or 9.5.

As used herein, the Expanded Disability Status Scale (EDSS) is a method of quantifying disability in multiple sclerosis and monitoring changes in the level of disability over time. It is widely used in clinical trials and in the assessment of people with MS. (See: Kurtzke J F. Rating neurologic impairment in multiple sclerosis: an expanded disability status scale (EDSS). Neurology. 1983 November; 33(11): 1444-52 and Haber A, LaRocca NG. eds. Minimal Record of Disability for multiple sclerosis. New York: National Multiple Sclerosis Society; 1985.)

The EDSS scale ranges from 0 to 10 in 0.5 unit increments that represent higher levels of disability. Scoring is based on an examination by a medical professional, usually a neurologist.

EDSS steps 1.0 to 4.5 refer to people with MS who are able to walk without any aid and is based on measures of impairment in eight functional systems (FS): pyramidal—weakness or difficulty moving limbs; cerebellar—ataxia, loss of coordination or tremor; brainstem—problems with speech, swallowing and nystagmus; sensory—numbness or loss of sensation; bowel and bladder function; visual function; cerebral (or mental) functions and other. Each functional system is scored on a scale of 0 (no disability) to 5 or 6 (more severe disability).

EDSS steps 5.0 to 9.5 are defined by the impairment to walking. Although the scale takes account of the disability associated with advanced MS, most people will never reach these scores.

Expanded Disability Status Scale (EDSS)

| Score | Description |
|---|---|
| 0.0 | Normal neurological exam (all grade 0 in |
| | all Functional System (FS) scores*) |
| 1.0 | No disability, minimal signs in one FS* |
| | (i.e., grade 1) |
| 1.5 | No disability, minimal signs in more |
| | than one FS* (more than 1 FS grade 1) |
| 2.0 | Minimal disability in one FS (one FS |
| | grade 2, others 0 or 1) |
| 2.5 | Minimal disability in two FS (two FS |
| | grade 2, others 0 or 1) |
| 3.0 | Moderate disability in one FS (one FS |
| | grade 3, others 0 or 1) or mild disability |
| | in three or four FS (three or four FS grade |
| | 2, others 0 or 1) though fully ambulatory |
| 3.5 | Fully ambulatory but with moderate |
| | disability in one FS (one grade 3) and |
| | one or two FS grade 2; or two FS grade 3 |
| | (others 0 or 1) or five grade 2 (others |
| | 0 or 1) |
| 4.0 | Fully ambulatory without aid, self- |
| | sufficient, up and about some 12 hours a |
| | day despite relatively severe disability |
| | consisting of one FS grade 4 (others 0 or |
| | 1), or combination of lesser grades |
| | exceeding limits of previous steps; able |
| | to walk without aid or rest some 500 meters |
| 4.5 | Fully ambulatory without aid, up and about |
| | much of the day, able to work a full day, |
| | may otherwise have some limitation of full |
| | activity or require minimal assistance; |
| | characterized by relatively severe |
| | disability usually consisting of one FS |
| | grade 4 (others or 1) or combinations of |
| | lesser grades exceeding limits of previous |
| | steps; able to walk without aid or rest |
| | some 300 meters. |
| 5.0 | Ambulatory without aid or rest for about |
| | 200 meters; disability severe enough to |
| | impair full daily activities (e.g., to |
| | work a full day without special provisions); |
| | (Usual FS equivalents are one grade 5 alone, |
| | others 0 or 1; or combinations of lesser |
| | grades usually exceeding specifications |
| | for step 4.0). |
| 5.5 | Ambulatory without aid for about 100 meters; |
| | disability severe enough to preclude full |
| | daily activities; (Usual FS equivalents are |
| | one grade 5 alone, others 0 or 1; or |
| | combination of lesser grades usually |
| | exceeding those for step 4.0) |
| 6.0 | Intermittent of unilateral constant |
| | assistance (cane, crutch, brace) required |
| | to walk about 100 meters with or without |
| | resting; (Usual FS equivalents are |
| | combinations with more than two FS grade 3+) |
| 6.5 | Constant bilateral assistance (canes, |
| | crutches, braces) required to walk about |
| | 20 meters without resting; (Usual FS |
| | equivalents are combinations with more than |
| | two FS grade 3+) |
| 7.0 | Unable to walk beyond approximately 5 |
| | meters even with aid, essentially restricted |
| | to wheelchair; wheels self in standard |
| | wheelchair and transfers alone; up and about |
| | in wheelchair some 12 hours a day; (Usual FS |
| | equivalents are combinations with more than |
| | one FS grade 4+; very rarely pyramidal grade |
| | 5 alone) |
| 7.5 | Unable to take more than a few steps. |
| | Restricted to wheelchair and may need aid |
| | in transferring. Can wheel self but cannot |
| | carry on in standard wheelchair for a full |
| | day and may require a motorized wheelchair; |
| | (Usual FS equivalents are combinations with |
| | more than one FS grade 4+) |
| 8.0 | Essentially restricted to bed or chair or |
| | perambulated in wheelchair. May be out of |
| | bed itself much of the day. Retains many |
| | self-care functions. Generally has effective |
| | use of arms; (Usual FS equivalents are |
| | combinations, generally 4+ in several |
| | systems) |
| 8.5 | Essentially restricted to bed much of day. |
| | Has some effective use of arms retains some |
| | self-care functions; (Usual FS equivalents |
| | are combinations, generally 4+ in several |
| | systems) |
| 9.0 | Confined to bed. Can still communicate and |
| | eat; (Usual FS equivalents are combinations, |
| | mostly grade 4+) |
| 9.5 | Confined to bed and totally dependent. |
| | Unable to communicate effectively or eat/ |
| | swallow; (Usual FS equivalents are |
| | combinations, almost all grade 4+) |
| 10 | Death due to MS |

*Excludes cerebral function grade 1

In certain embodiments, the subject is human. In further such embodiments, the subject is a female human.

Demyelination of axons in chronic MS can result in axon degeneration and neuronal cell death, but more specifically, MS destroys oligodendrocytes, the highly specialized CNS cells that generate and maintain myelin.

Neuromyelitis Optica (NMO), is also referred to as Devic's disease. NMO is a disorder of the central nervous system (CNS) that predominantly affects the optic nerve and spinal cord of patients.

Leukodystrophies are a group of progressive, metabolic, genetic diseases that affect the brain, spinal cord and often the peripheral nerves. Each type of leukodystrophy is caused by a specific gene abnormality that leads to abnormal development or destruction of the myelin sheath of the brain. Each type of leukodystrophy affects a different part of the myelin sheath, leading to a range of neurological problems. Exemplary leukodystrophies which may be treated or ameliorated by the methods of the present invention include, but are not limited to, adult-onset autosomal dominant leukodystrophy (ADLD), Aicardi-Goutieres syndrome, Alexander disease, CADASIL, Canavan disease, CARASIL, cerebrotendionous xanthomatosis, childhood ataxia and cerebral hypomyelination (CACH)/vanishing white matter disease (VWMD), Fabry disease, fucosidosis, GM1 gangliosidosis, Krabbe disease, L-2-hydroxyglutaric aciduria, megalencephalic leukoencephalopathy with subcortical cysts, metachromatic leukodystrophy, multiple sulfatase deficiency, Pelizaeus-Merzbacher disease (PMD), Pol III-related leukodystrophies, Refsum disease, salla disease (free sialic acid storage disease), Sjogren-Larsson syndrome, X-linked adrenoleukodystrophy, and Zellweger syndrome spectrum disorders.

Myelin-related disorders which can be treated or ameliorated by the methods of the present invention include a disorder characterized by a myelin deficiency. Insufficient myelination in the central nervous system has been implicated in a wide array of neurological disorders. Among these are forms of cerebral palsy wherein a congenital deficit in forebrain myelination in children with periventricular leukomalacia, contributes to neurological morbidity (Goldman et al., 2008) Goldman, S. A., Schanz, S., and Windrem, Mi S. (2008). Stem cell-based strategies for treating pediatric disorders of myelin. Hum Mol Genet. 17, R76-83. At the other end of the age spectrum, myelin loss and ineffective repair may contribute to the decline in cognitive function associated with senescence (Kohama et al., 2011) Kohama, S. G., Rosene, D. L., and Sherman, L. S. (2011) Age (Dordr). Age-related changes in human and non-human primate white matter: from myelination disturbances to cognitive decline. Therefore, it is contemplated that effective compounds and methods of enhancing myelination and/or remyelination may have substantial therapeutic benefits in halting disease progression and restoring function in MS and in a wide array of neurological disorders.

Myelination of neurons requires oligodendrocytes. The term “myelination”, as used herein, refers to the generation of the nerve's myelin sheath by replacing myelin producing cells or restoring their function. The neurons that undergo remyelination may be in the brain, spinal cord, or both the brain and spinal cord.

“Promoting Myelination” as used herein refers to increasing the rate of myelin production rather than a mere net increase in the amount of myelin as compared to a baseline level of myelin production rate in a subject. An increase in the rate of myelin production can be determined using imaging techniques or functional measurements.

A “baseline level of myelin production rate” as used herein, refers to the rate of myelin production in subject being treated before the onset of treatment.

“MS therapeutic agents” as used herein, refers to therapeutic agents known to be used in treating MS. Such therapeutic agents include, but are not limited to, Copaxone (glatiramer acetate), Ocrevus (ocrelizumab), Campath (Lemtrada or alemtuzumab), Gilenya, Ampyra (dalfampridine), Tysabri (natalizumab), Aubagio (teriflunomide), Rebif, Avonex, Betaseron, Plegridy, Interferon Beta-la, dimethyl fumarate, fingolimod, rituximab, Zinbryta, Ofatumymab, Nerventra (laquinimod), Masitinib, Siponimod, Ozanimod, Ponesimod, ibudilast, vatelizumab, minocycline, ibrutinib, PRN2246, Cladripine, GNBAC1, daclizumab, and MD1003 (biotin).

In certain embodiments, the compounds of the invention and pharmaceutically acceptable salts or solvates thereof are administered in combination with a therapeutically effective amount of an MS therapeutic agent.

The MS therapeutic agent may be administered simultaneously with the compound of the invention. Alternatively, the MS therapeutic agent may be administered prior to administration the compound of the invention. Alternatively still, the MS therapeutic agent may be administered following the administration of the compound of the invention.

In certain embodiments, the compounds of the invention and pharmaceutically acceptable solvates thereof can be administered in combination with cognitive enhancing (nootropic) agents. Exemplary agents include any drugs, supplements, or other substances that improve cognitive function, particularly executive functions, memory, creativity, or motivation, in healthy individuals. Non-limiting examples include racetams (e.g., piracetam, oxiracetam, and aniracetam), nutraceuticals (e.g., bacopa monnieri, panax ginseng, ginko biloba, and GABA), stimulants (e.g., amphetamine pharmaceuticals, methylphenidate, eugeroics, xanthines, and nicotine), L-Theanine, Tolcapone, Levodopa, Atomoxetine, and Desipramine.

A further embodiment for treating a subject suffering from a myelin-related disorder is to administer a therapeutically effective amount of a compound described herein along with a therapeutically effective amount of additional oligodendrocyte differentiation and/or proliferation inducing agent(s) and/or anti-neurodegenerative disease agent. Examples of anti-neurodegenerative disease agents include L-dopa, cholinesterase inhibitors, anticholinergics, dopamine agonists, steroids, immunomodulators including interferons, monoclonal antibodies, and glatiramer acetate and modulators (e.g., inhibitors) of SARMI a new class of NADase enzyme (See, Essuman, Neuron, Vol. 93, Issue 6, pa 1334, Mar. 22, 2017).

Therefore, in a further aspect of the invention, the oligodendrocyte precursor differentiation and/or proliferation inducing compound of formula II or III described herein can be administered as part of a combination therapy with adjunctive therapies for treating neurodegenerative and myelin related disorders.

The phrase “combination therapy” embraces the administration of the crystalline form of the compound of formula II or III and an additional therapeutic agent as part of a specific treatment regimen intended to provide a beneficial effect from the co-action of each. When administered as a combination, the oligodendrocyte precursor differentiation inducing compound (the crystalline form of the compound of formula II or III) and an additional therapeutic agent can be formulated as separate compositions. Administration of these therapeutic agents in combination typically is carried out over a defined time period (usually minutes, hours, days or weeks depending upon the combination selected).

“Combination therapy” is intended to embrace administration of these therapeutic agent (the crystalline form of the compound of formula II or III and an additional therapeutic agent) in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. The sequence wherein the therapeutic agents are administered is not narrowly critical. “Combination therapy” also can embrace the administration of the therapeutic agents as described above in further combination with other biologically active ingredients (such as, but not limited to, a second and different therapeutic agent) and non-drug therapies (e.g., surgery).

In another aspect of the invention, the therapeutic agents administered in a combination therapy with the oligodendrocyte differentiation and/or proliferation inducing compound described herein (the crystalline form of the compound of formula II or III) can include at least one anti-neurodegenerative agent such as but not limited to, an immunotherapeutic agent.

In certain embodiments of the method described herein (promoting myelination of central nervous system axons in a subject suffering from a myelin-related disorder) the subject is administered the compound of formula I for an on-drug cycle of at least three months. For example, the subject is administered the crystalline form of the compound of formula II or III for an on-drug cycle of at least six months.

In particular embodiments of the method described herein (promoting myelination of central nervous system axons in a subject suffering from a myelin-related disorder), the subject is administered the crystalline form of the compound of formula II or III using the following dosing regimen: on-drug cycle for at least six months; off-drug cycle for at least three months; wherein the on-drug and off-drug cycles are optionally repeated.

### Exemplification

### General Methods and Materials

### XRPD

The data presented in this application contain x-ray diffraction patterns with labeled peaks and tables with peak lists. The range of data collected is instrument dependent. Under most circumstances, peaks within the range of up to about 30° 2θ were selected. Rounding algorithms were used to round each peak to the nearest 0.1° or 0.01° 2θ, depending upon the instrument used to collect the data and/or the inherent peak resolution. The location of the peaks along the x-axis (° 2θ) in both the FIGS. and the tables were determined using proprietary software and rounded to one or two significant FIGS. after the decimal point based upon the above criteria. Peak position variabilities are given to within 0.2° 20 based upon recommendations outlined in the USP discussion of variability in x-ray powder diffraction (United States Pharmacopeia, USP 38-NF 33 through Si, <941>8/1/2015). For d-space listings, the wavelength used to calculate d-spacings was 1.5405929 Å, the Cu—K_{α1} wavelength (Phys. Rev. A56 (6) 4554-4568 (1997)). Variability associated with d-spacing estimates was calculated from the USP recommendation, at each d-spacing, and provided in the respective tables.

Per USP guidelines, variable hydrates and solvates may display peak variances greater than 0.2° 2θ and therefore peak variances of 0.2° 2θ are not applicable to these materials.

“Prominent Peaks” are a subset of the entire observed peak list. Prominent peaks are selected from observed peaks by identifying preferably non-overlapping, low-angle peaks, with strong intensity.

If multiple diffraction patterns are available, then assessments of particle statistics (PS) and/or preferred orientation (PO) are possible. Reproducibility among XRPD patterns from multiple samples analyzed on a single diffractometer indicates that the particle statistics are adequate. Consistency of relative intensity among XRPD patterns from multiple diffractometers indicates good orientation statistics. Alternatively, the observed XRPD pattern may be compared with a calculated XRPD pattern based upon a crystal structure, if available. Two-dimensional scattering patterns using area detectors can also be used to evaluate PS/PO. If the effects of both PS and PO are determined to be negligible, then the XRPD pattern is representative of the powder average intensity for the sample and prominent peaks may be identified as “Representative Peaks.” In general, the more data collected to determine Representative Peaks, the more confident one can be of the classification of those peaks.

“Characteristic peaks,” to the extent they exist, are a subset of Representative Peaks and are used to differentiate one crystalline polymorph from another crystalline polymorph (polymorphs being crystalline forms having the same chemical composition). Characteristic peaks are determined by evaluating which representative peaks, if any, are present in one crystalline polymorph of a compound against all other known crystalline polymorphs of that compound to within ±0.2° 2θ. Not all crystalline polymorphs of a compound necessarily have at least one characteristic peak.

XRPD patterns were collected with a PANalytical X'Pert PRO MPD diffractometer using an incident beam of Cu radiation produced using an Optix long, fine-focus source. An elliptically graded multilayer mirror was used to focus Cu Kα X-ray radiation through the specimen and onto the detector. Prior to the analysis, a silicon specimen (NIST SRM 640e) was analyzed to verify the observed position of the Si (111) peak is consistent with the NIST-certified position. A specimen of the sample was sandwiched between 3-μm-thick films and analyzed in transmission geometry. A beam-stop, short antiscatter extension, and antiscatter knife edge, were used to minimize the background generated by air. Soller slits for the incident and diffracted beams were used to minimize broadening from axial divergence. Diffraction patterns were collected using a scanning position-sensitive detector (X'Celerator) located 240 mm from the specimen and Data Collector software v. 2.2b.

### TGA

TG analyses described herein were performed using a Mettler Toledo TGA/DSC3+ analyzer. Balance check was performed using calcium oxalate, and temperature calibration was performed using indium, tin, and zinc. The sample was placed in an aluminum pan. The sample was sealed, the lid pierced, then inserted into the TG furnace. The furnace was heated under nitrogen.

### DSC

DSC analyses described herein were performed using a Mettler-Toledo DSC3+ differential scanning calorimeter. Temperature calibration was performed using octane, phenyl salicylate, indium, tin, and zinc. The samples were placed into aluminum DSC pans, covered with lids, and the weights were accurately recorded. A weighed aluminum pan conFIG.d as the sample pan was placed on the reference side of the cell. The pan lids were pierced prior to sample analysis.

### DVS

DVS data were collected on a VTI SGA-100 Vapor Sorption Analyzer. NaCl and PVP were used as calibration standards. Samples were not dried prior to analysis. Sorption and desorption data were collected over a range from 5% to 95% RH at 10% RH increments under a nitrogen purge. The equilibrium criterion used for analysis was less than 0.0100% weight change in 5 minutes with a maximum equilibration time of 3 hours. Data were not corrected for the initial moisture content of the samples.

### Kinetic Aqueous Solubilides

Weighed samples of material were treated with aliquots of water at ambient temperature. Samples were typically sonicated between additions to facilitate dissolution. Complete dissolution was observed through visual inspection. Solubilities were calculated based on the total amount of solvent added to achieve complete dissolution and may be greater than the value reported due to incremental solvent addition and the inherent kinetics of dissolution. If dissolution was not observed, values are reported as “less than”. If complete dissolution was observed upon the first aliquot of solvent, values are reported as “greater than”.

| Abbreviation | Meaning |
|---|---|
| DEE | diethyl ether |
| EtOAc. EA | ethyl acerate |
| EtOH | ethanol |
| h, hr | hour |
| H₂O | water |
| min | minute |
| MeOH | methanol |
| MeCN | acetonitrile |
| MTBE | methyl tert-butyl ether |
| MEK | methyl ethyl ketone |
| IPE | diisopropyl ether |

### Example 1—Esylate Salt of Cis-N-cyclohexyl-N-ethyl-3-(3-chloro-4-cyclohexylphenyl)prop-2-enylamine (Compound of Formula II)

Method 1: cis-N-cyclohexyl-N-ethyl-3-(3-chloro-4-cyclohexylphenyl)prop-2-enylamine (69.5 mg) was dissolved in anhydrous EtOAc (500 μL), and ethanesulfonic acid (17.5 μL) was added. The solution was stirred for approximately 1 day on a magnetic stirrer. Heptane and diethyl ether were added, and mixture was left uncapped at ambient temperature for fast evaporation, resulting in a gel formation. The gel was then broken up, MTBE was added, and the mixture was stirred at ambient temperature for ˜7 days. Solids produced were isolated by vacuum filtration. Removal of residual solvent under high vacuum yielded the salt.

Method 2: cis-N-cyclohexyl-N-ethyl-3-(3-chloro-4-cyclohexylphenyl)prop-2-enylamine (84.3 mg) was dissolved in anhydrous EtOAc (500 μL), and ethanesulfonic acid (21 μL) was added. The solution was stirred for approximately 5 days on a magnetic stirrer. Solids produced were isolated by vacuum filtration. Removal of residual solvent under high vacuum yielded the salt.

The TGA trace of the crystalline form of compound of formula II is depicted in FIG. 2. Weight loss of approximately 0.9% from ambient to 93° C. was observed in the TG thermogram. Large weight loss with onset at 268° C. corresponds to decomposition.

The DSC trace of the crystalline form of compound of formula II in is depicted FIG. 3. The DSC thermogram displays a small broad endothermic peak at approximately 54° C. and a large endothermic peak at 138° C.

The DVS pattern of the crystalline form of compound of formula II is depicted in FIG. 4. The DVS pattern displays a weight gain of about 0.35% at 5-85% RH and of 0.51% at 85-95% RH.

The crystalline form of compound of formula II is a monoclinic unit cell, space group P2₁/c, with unit cell parameters:

a=12.652 Å α=90°
b=19.013 Å β=91.97°
c=10.583 Å γ=90°
unit cell volume=2,544.3 Å³

The XRPD patterns of the crystalline form of compound of formula II is depicted in FIG. 1, and the peaks are tabulated in Table 3.

TABLE 3

| °2θ | d space (Å) | Intensity (%) |
|---|---|---|
| 6.97 ± | 12.677 ± | 39 |
| 0.20 | 0.363 | |
| 8.38 ± | 10.547 ± | 9 |
| 0.20 | 0.251 | |
| 9.28 ± | 9.522 ± | 22 |
| 0.20 | 0.205 | |
| 9.54 ± | 9.264 ± | 5 |
| 0.20 | 0.194 | |
| 11.65 + | 7.587 ± | 13 |
| 0.20 | 0.130 | |
| 12.03 ± | 7.353 ± | 18 |
| 0.20 | 0.122 | |
| 12.50 ± | 7.077 ± | 80 |
| 0.20 | 0.113 | |
| 14.00 ± | 6.322 ± | 56 |
| 0.20 | 0.090 | |
| 14.48 ± | 6.111 ± | 10 |
| 0.20 | 0.084 | |
| 14.75 ± | 5.999 ± | 34 |
| 0.20 | 0.081 | |
| 15.63 ± | 5.665 ± | 9 |
| 0.20 | 0.072 | |
| 16.73 ± | 5.296 ± | 73 |
| 0.20 | 0.063 | |
| 17.38 ± | 5.099 ± | 19 |
| 0.20 | 0.048 | |
| 17.63 ± | 5.027 ± | 19 |
| 0.20 | 0.057 | |
| 17.87 ± | 4.959 ± | 65 |
| 0.20 | 0.055 | |
| 18.65 ± | 4.753 ± | 44 |
| 0.20 | 0.051 | |
| 18.98 ± | 4.671 ± | 35 |
| 0.20 | 0.049 | |
| 19.83 ± | 4.473 ± | 26 |
| 0.20 | 0.045 | |
| 20.47 ± | 4.334 ± | 100 |
| 0.20 | 0.042 | |
| 21.50 ± | 4.129 ± | 34 |
| 0.20 | 0.038 | |
| 21.79 ± | 4.076 ± | 23 |
| 0.20 | 0.037 | |
| 22.29 ± | 3.986 ± | 19 |
| 0.20 | 0.035 | |
| 22.82 ± | 3.894 ± | 14 |
| 0.20 | 0.034 | |
| 23.42 ± | 3.795 ± | 29 |
| 0.20 | 0.032 | |
| 24.31 ± | 3.658 ± | 12 |
| 0.20 | 0.030 | |
| 24.80 ± | 3.587 ± | 21 |
| 0.20 | 0.028 | |
| 25.18 ± | 3.534 ± | 16 |
| 0.20 | 0.028 | |
| 25.38 ± | 3.506 ± | 17 |
| 0.20 | 0.027 | |
| 25.68 ± | 3.467 ± | 14 |
| 0.20 | 0.027 | |
| 25.98 ± | 3.427 ± | 7 |
| 0.20 | 0.026 | |
| 26.41 ± | 3.372 ± | 14 |
| 0.20 | 0.025 | |
| 26.97 ± | 3.304 ± | 20 |
| 0.20 | 0.024 | |

### Example 2—Salicylate Salt of Cis-N-cyclohexyl-N-ethyl-3-(3-chloro-4-cyclohexylphenyl)prop-2-enylamine (Compound of Formula III)

Method 1: cis-N-cyclohexyl-N-ethyl-3-(3-chloro-4-cyclohexylphenyl)prop-2-enylamine (56.3 mg) was dissolved in IPE (1 mL). Salicylic acid (20.5 mg) was dissolved in EtOH (500 μL) and added to the solution of cis-N-cyclohexyl-N-ethyl-3-(3-chloro-4-cyclohexylphenyl)prop-2-enylamine. Additional IPE was added (1 mL) to the mixture, which was then stirred at ambient temperature for approximately 4 days (precipitation was observed within a few minutes of the IPE addition). Solids produced were isolated by vacuum filtration. Removal of residual solvent under high vacuum yielded the salt.

Method 2: cis-N-cyclohexyl-N-ethyl-3-(3-chloro-4-cyclohexylphenyl)prop-2-enylamine (70.1 mg) was dissolved in IPE (1 mL). Salicylic acid (29.1 mg) was dissolved in EtOH (500 μL) and added to the solution of cis-N-cyclohexyl-N-ethyl-3-(3-chloro-4-cyclohexylphenyl)prop-2-enylamine. Precipitation was observed within a few minutes. The suspension was stirred at ambient temperature for approximately 5 days. Solids produced were isolated by vacuum filtration. Removal of residual solvent under high vacuum yielded the salt. The XRPD pattern was consistent with salicylate salt obtained by Method 1, containing small additional peaks at 8.4 and 15.1° 2θ.

The TGA trace of the crystalline form of compound of formula III is depicted in FIG. 6. Large weight loss with onset at 212° C. corresponds to decomposition.

The DSC trace of the crystalline form of compound of formula III is depicted in FIG. 7. The DSC thermogram displays a large endothermic peak at 137° C.

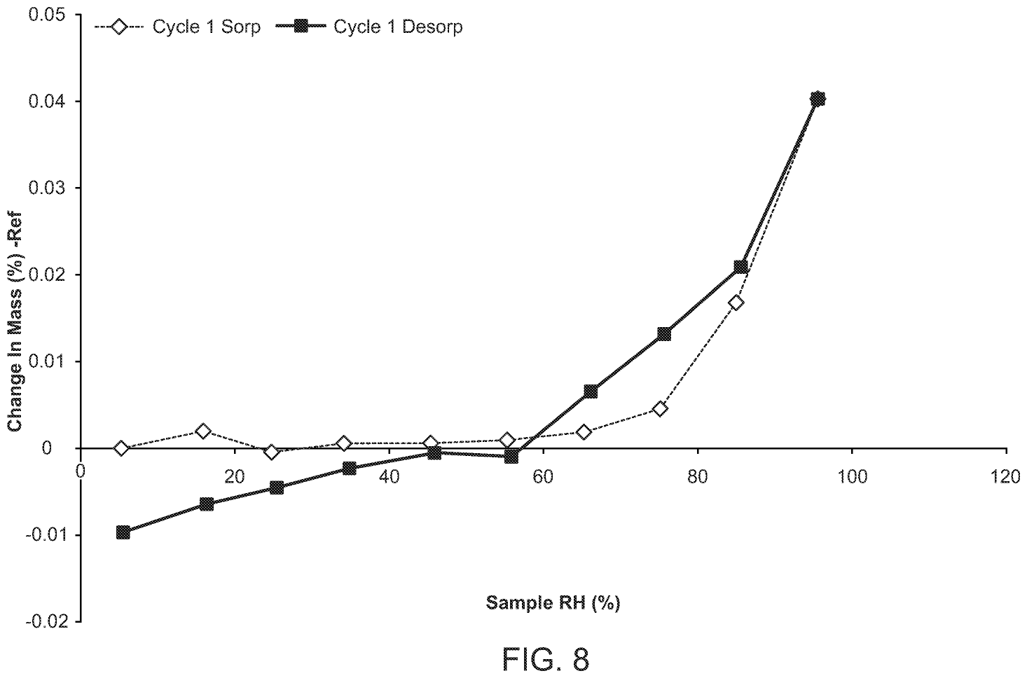

The DVS pattern of the crystalline form of compound of formula III is depicted in FIG. 8. The DVS pattern displays a weight gain of about 0.04% at 5-95% RH.

The crystalline form of compound of formula III is a monoclinic unit cell, space group P2₁/c, with unit cell parameters:

a=18.958 Å α=90°
b=10.910 Å β=109.44°
c=14.445 Å γ=90°
unit cell volume=2,817.3 Å³

The XRPD patterns of the crystalline form of compound of formula III is depicted in FIG. 5, and the peaks are tabulated in Table 4.

TABLE 4

| °2θ | d space (Å) | Intensity (%) |
|---|---|---|
| 4.93 ± | 17.895 ± | 40 |
| 0.20 | 0.725 | |
| 9.48 ± | 9.326 ± | 8 |
| 0.20 | 0.196 | |
| 10.38 ± | 8.516 ± | 9 |
| 0.20 | 0.164 | |
| 10.52 ± | 8.404 ± | 14 |
| 0.20 | 0.159 | |
| 12.26 ± | 7.215 ± | 7 |
| 0.20 | 0.117 | |
| 12.40 ± | 7.134 ± | 6 |
| 0.20 | 0.115 | |
| 12.79 ± | 6.917 ± | 29 |
| 0.20 | 0.108 | |
| 12.98 ± | 6.813 ± | 22 |
| 0.20 | 0.104 | |
| 13.45 ± | 6.579 ± | 20 |
| 0.20 | 0.097 | |
| 14.71 ± | 6.016 ± | 100 |
| 0.20 | 0.081 | |
| 14.86 ± | 5.956 ± | 16 |
| 0.20 | 0.080 | |
| 15.33 ± | 5.776 ± | 11 |
| 0.20 | 0.075 | |
| 15.79 ± | 5.610 ± | 24 |
| 0.20 | 0.071 | |
| 16.24 ± | 5.453 ± | 14 |
| 0.20 | 0.067 | |
| 16.97 ± | 5.220 ± | 27 |
| 0.20 | 0.061 | |
| 17.39 ± | 5.095 ± | 25 |
| 0.20 | 0.058 | |
| 17.59 ± | 5.039 ± | 26 |
| 0.20 | 0.057 | |
| 18.11 ± | 4.894 ± | 11 |
| 0.20 | 0.054 | |
| 19.03 ± | 4.659 ± | 32 |
| 0.20 | 0.048 | |
| 19.79 ± | 4.484 ± | 17 |
| 0.20 | 0.045 | |
| 20.21 ± | 4.391 ± | 6 |
| 0.20 | 0.043 | |
| 20.42 ± | 4.345 ± | 22 |
| 0.20 | 0.042 | |
| 20.85 ± | 4.258 ± | 70 |
| 0.20 | 0.040 | |
| 21.18 ± | 4.191 ± | 20 |
| 0.20 | 0.039 | |
| 21.48 ± | 4.134 ± | 9 |
| 0.20 | 0.038 | |
| 21.84 ± | 4.066 ± | 8 |
| 0.20 | 0.037 | |
| 22.08 ± | 4.023 ± | 27 |
| 0.20 | 0.036 | |
| 22.44 ± | 3.960 ± | 5 |
| 0.20 | 0.035 | |
| 22.86 ± | 3.887 ± | 8 |
| 0.20 | 0.034 | |
| 22.98 ± | 3.868 ± | 9 |
| 0.20 | 0.033 | |
| 23.94 ± | 3.714 ± | 4 |
| 0.20 | 0.031 | |
| 24.26 ± | 3.666 ± | 11 |
| 0.20 | 0.030 | |
| 24.94 ± | 3.567 ± | 33 |
| 0.20 | 0.028 | |
| 25.39 ± | 3.505 ± | 51 |
| 0.20 | 0.027 | |
| 26.11 ± | 3.410 ± | 7 |
| 0.20 | 0.026 | |
| 26.23 ± | 3.394 ± | 9 |
| 0.20 | 0.025 | |
| 26.43 ± | 3.369 ± | 9 |
| 0.20 | 0.025 | |
| 27.26 ± | 3.269 ± | 3 |
| 0.20 | 0.024 | |
| 28.02 ± | 3.182 ± | 9 |
| 0.20 | 0.022 | |
| 28.34 ± | 3.147 ± | 10 |
| 0.20 | 0.022 | |
| 29.02 ± | 3.074 ± | 3 |
| 0.20 | 0.021 | |
| 29.96 ± | 2.980 ± | 13 |
| 0.20 | 0.019 | |

### Example 3—Besylate Salt of Cis-N-cyclohexyl-N-ethyl-3-(3-chloro-4-cyclohexylphenyl)prop-2-enylamine

cis-N-cyclohexyl-N-ethyl-3-(3-chloro-4-cyclohexylphenyl)prop-2-enylamine (133.4 mg) was dissolved in anhydrous EtOAc (1 mL), and solids of benzenesulfonic acid (66.8 mg) were added. The mixture was stirred using a magnetic stirrer at ambient temperature for approximately 1 day, at which point heptane and DEE were added and agitated. Formation of a gel was observed. The solidified gel was then broken up, and the suspension was stirred for approximately 4 days. Solids were isolated by vacuum filtration. Removal of residual solvent under high vacuum yielded the salt.

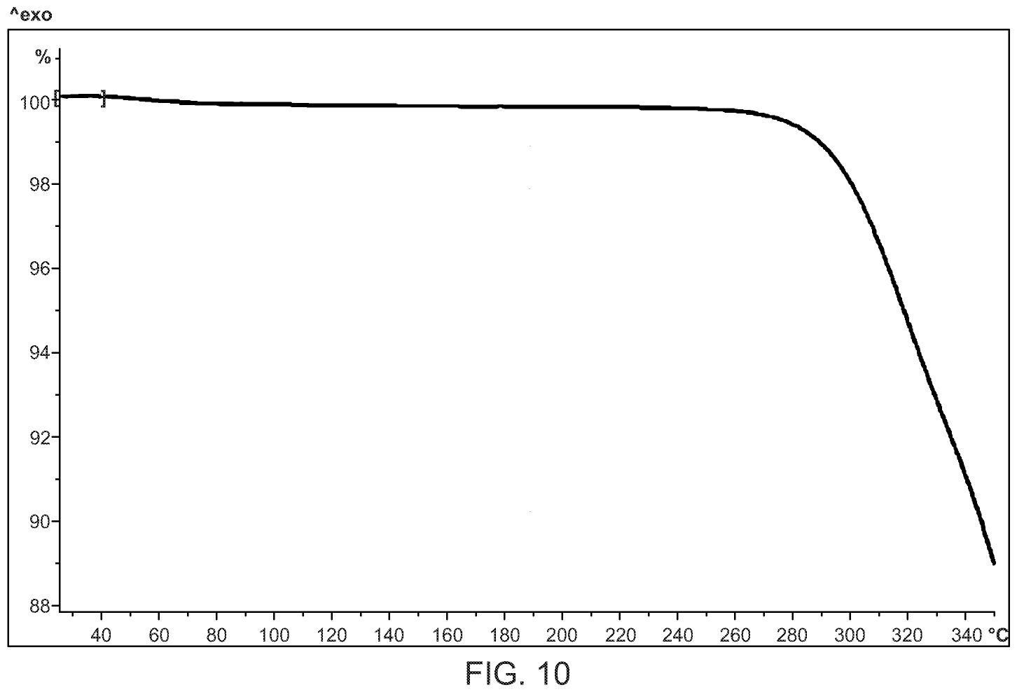

The TGA trace of the crystalline form of the besylate salt of cis-N-cyclohexyl-N-ethyl-3-(3-chloro-4-cyclohexylphenyl)prop-2-enylamine is depicted in FIG. 10. Weight loss of approximately 0.2% from 30° C. to 79° C. was observed in the TG thermogram. Large weight loss with onset at 274° C. corresponds to decomposition.

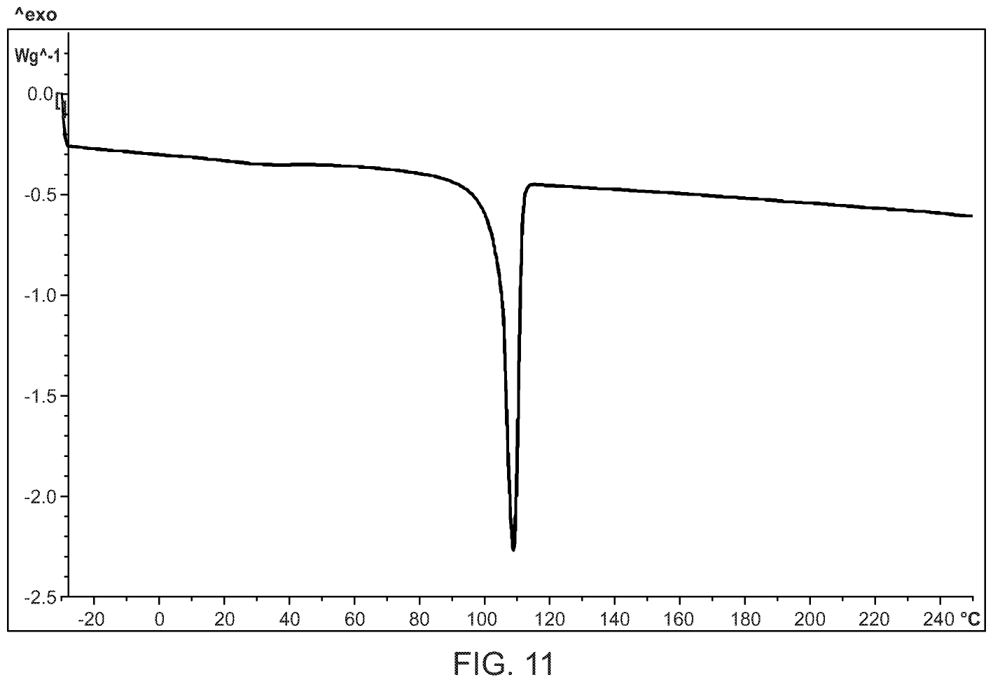

The DSC trace of the crystalline form of the besylate salt of cis-N-cyclohexyl-N-ethyl-3-(3-chloro-4-cyclohexylphenyl)prop-2-enylamine is depicted in FIG. 11. The DSC thermogram displays a large endothermic peak at 108° C.

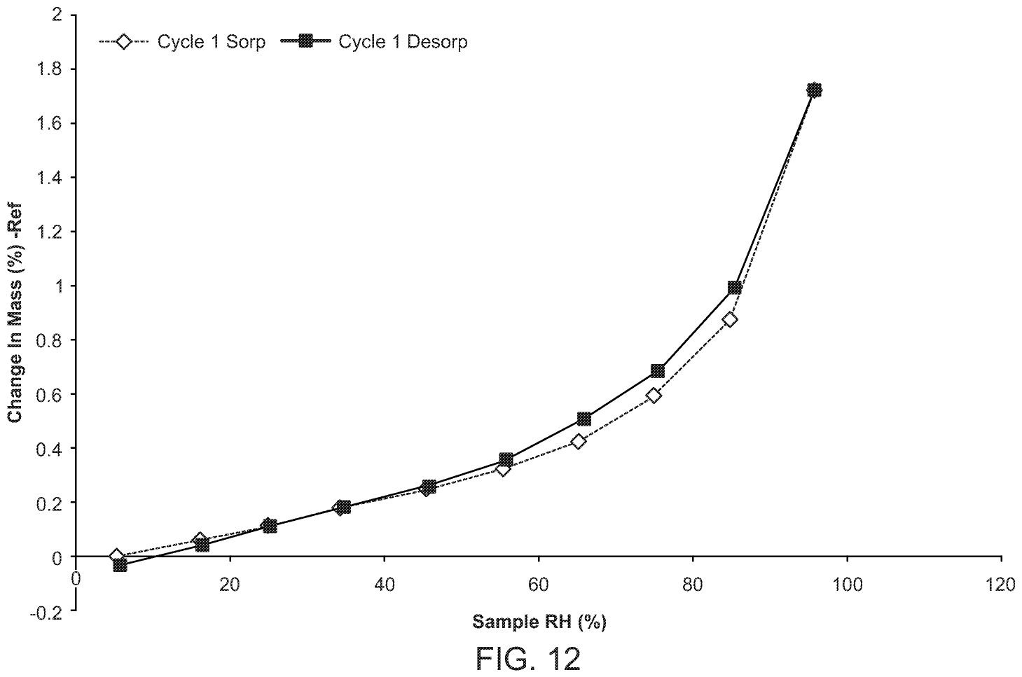

The DVS pattern of the crystalline form of the besylate salt of cis-N-cyclohexyl-N-ethyl-3-(3-chloro-4-cyclohexylphenyl)prop-2-enylamine is depicted in FIG. 12. The DVS pattern displays a weight gain of about 0.88% at 5-85% RH and of about 0.85% at 85-95% RH.

The crystalline form of the besylate salt of cis-N-cyclohexyl-N-ethyl-3-(3-chloro-4-cyclohexylphenyl)prop-2-enylamine is an orthorhombic unit cell, space group Pna2₁, with unit cell parameters:

a=11.495 Å α=90°
b=9.051 Å β=90°
c=27.333 Å γ=90°
unit cell volume=2,843.8 Å³

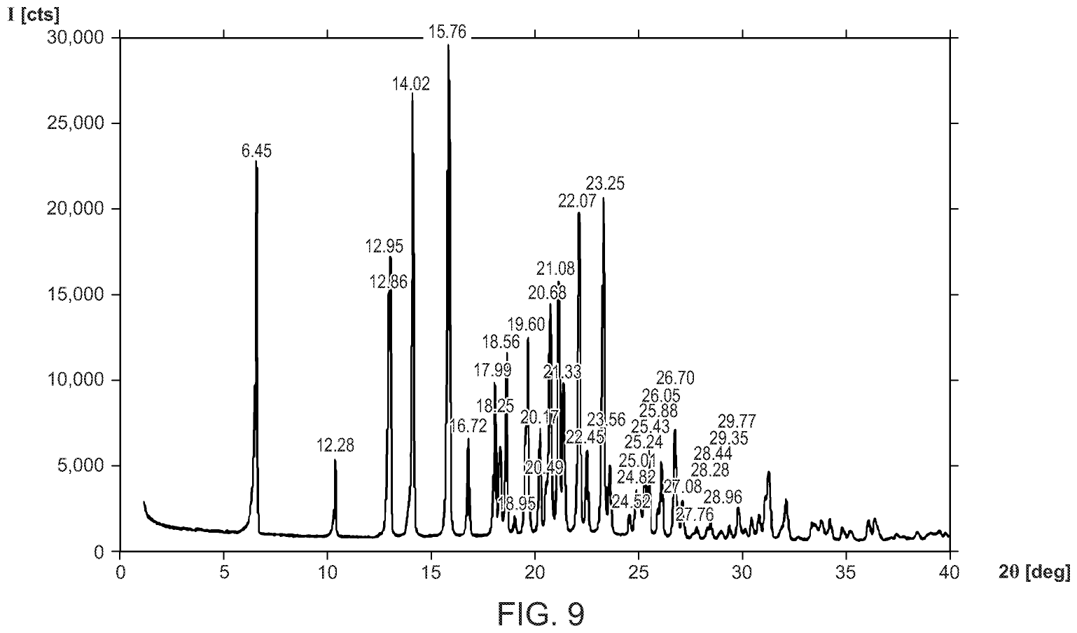

The XRPD patterns of the crystalline form of compound of formula II is depicted in FIG. 9, and the peaks are tabulated in Table 5.

TABLE 5

| °2θ | d space (Å) | Intensity (%) |
|---|---|---|
| 6.45 ± | 13.693 ± | 75 |
| 0.20 | 0.424 | |
| 10.28 ± | 8.601 ± | 18 |
| 0.20 | 0.167 | |
| 12.86 ± | 6.880 ± | 50 |
| 0.20 | 0.107 | |
| 12.95 ± | 6.833 ± | 58 |
| 0.20 | 0.105 | |
| 14.02 ± | 6.311 ± | 90 |
| 0.20 | 0.090 | |
| 15.76 ± | 5.619 ± | 100 |
| 0.20 | 0.071 | |
| 16.72 ± | 5.299 ± | 22 |
| 0.20 | 0.063 | |
| 17.99 ± | 4.927 ± | 33 |
| 0.20 | 0.054 | |
| 18.25 ± | 4.856 ± | 20 |
| 0.20 | 0.053 | |
| 18.56 ± | 4.778 ± | 39 |
| 0.20 | 0.051 | |
| 18.95 ± | 4.679 ± | 7 |
| 0.20 | 0.049 | |
| 19.60 ± | 4.526 ± | 42 |
| 0.20 | 0.046 | |
| 20.17 ± | 4.399 ± | 24 |
| 0.20 | 0.043 | |
| 20.49 ± | 4.331 ± | 14 |
| 0.20 | 0.042 | |
| 20.68 ± | 4.291 ± | 48 |
| 0.20 | 0.041 | |
| 21.08 ± | 4.211 ± | 53 |
| 0.20 | 0.040 | |
| 21.33 ± | 4.162 ± | 33 |
| 0.20 | 0.039 | |
| 22.07 ± | 4.024 ± | 67 |
| 0.20 | 0.036 | |
| 22.45 ± | 3.956 ± | 20 |
| 0.20 | 0.035 | |
| 23.25 ± | 3.823 ± | 70 |
| 0.20 | 0.032 | |
| 23.56 ± | 3.773 ± | 17 |
| 0.20 | 0.032 | |
| 24.52 ± | 3.628 ± | 7 |
| 0.20 | 0.029 | |
| 24.82 ± | 3.584 ± | 12 |
| 0.20 | 0.028 | |
| 25.01 ± | 3.558 ± | 10 |
| 0.20 | 0.028 | |
| 25.24 ± | 3.526 ± | 15 |
| 0.20 | 0.027 | |
| 25.43 ± | 3.500 ± | 21 |
| 0.20 | 0.027 | |
| 25.88 ± | 3.440 ± | 8 |
| 0.20 | 0.026 | |
| 26.05 ± | 3.417 ± | 18 |
| 0.20 | 0.026 | |
| 26.70 ± | 3.336 ± | 24 |
| 0.20 | 0.025 | |
| 27.08 ± | 3.290 ± | 10 |
| 0.20 | 0.024 | |
| 27.76 ± | 3.211 ± | 5 |
| 0.20 | 0.023 | |
| 28.28 ± | 3.153 ± | 5 |
| 0.20 | 0.022 | |
| 28.44 ± | 3.135 ± | 6 |
| 0.20 | 0.022 | |
| 28.96 ± | 3.081 ± | 4 |
| 0.20 | 0.021 | |
| 29.35 ± | 3.041 ± | 5 |
| 0.20 | 0.020 | |
| 29.77 ± | 2.998 ± | 8 |
| 0.20 | 0.020 | |

### Example 4—Xinafoate Salt of Cis-N-cyclohexyl-N-ethyl-3-(3-chloro-4-cyclohexylphenyl)prop-2-enylamine

cis-N-cyclohexyl-N-ethyl-3-(3-chloro-4-cyclohexylphenyl)prop-2-enylamine (77.3 mg) was dissolved in IPE (1 mL). Xinafoic acid (52.4 mg) was dissolved in MEK (500 μL) and added to the solution of cis-N-cyclohexyl-N-ethyl-3-(3-chloro-4-cyclohexylphenyl)prop-2-enylamine. The mixture was stirred using a magnetic stirrer at ambient temperature for approximately 4 days. Solids produced were isolated by vacuum filtration. Removal of residual solvent under high vacuum yielded the salt.

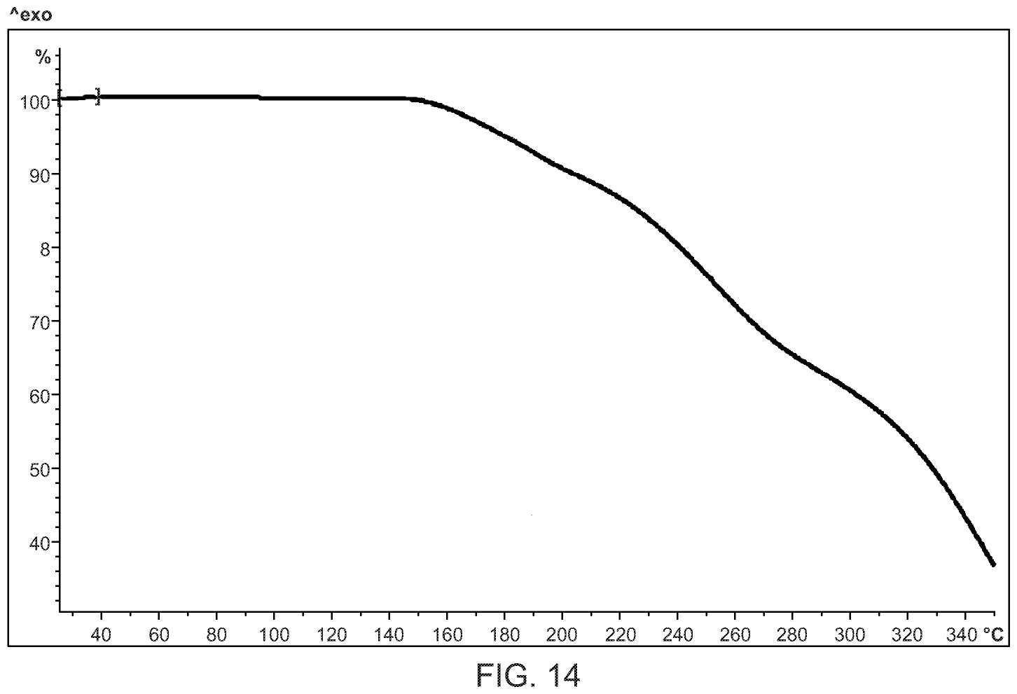

The TGA trace of the crystalline form of xinafoate salt of cis-N-cyclohexyl-N-ethyl-3-(3-chloro-4-cyclohexylphenyl)prop-2-enylamine is depicted in FIG. 14. Weight loss of approximately 0.1% from 37° C. to 117° C. was observed in the TG thermogram. Large weight loss with onset at 146° C. corresponds to decomposition.

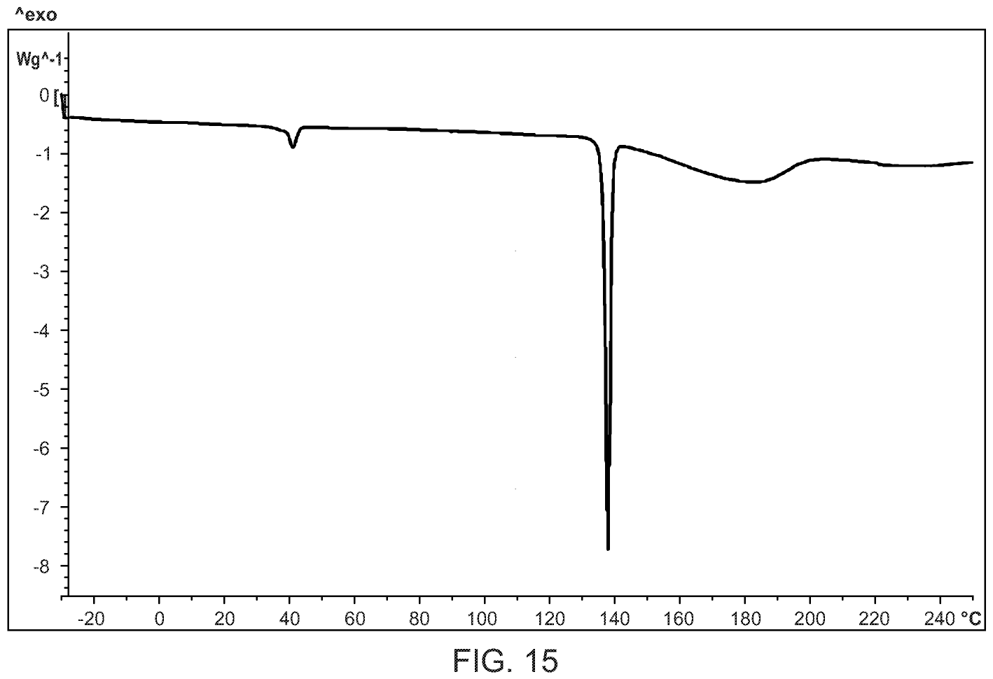

The DSC trace of the crystalline form of the crystalline form of xinafoate salt of cis-N-cyclohexyl-N-ethyl-3-(3-chloro-4-cyclohexylphenyl)prop-2-enylamine is depicted in FIG. 15. The DSC thermogram displays a small endothermic peak at 41° C. and a large endothermic peak at 137° C.

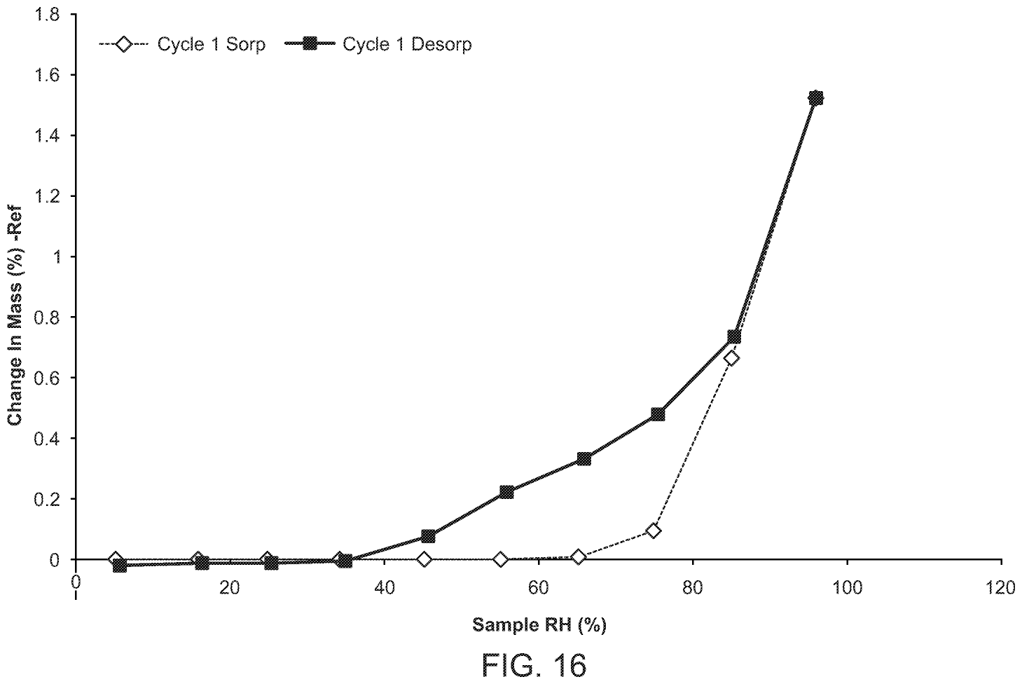

The DVS pattern of the crystalline form of xinafoate salt of cis-N-cyclohexyl-N-ethyl-3-(3-chloro-4-cyclohexylphenyl)prop-2-enylamine is depicted in FIG. 16. The DVS pattern displays a weight gain of about 0.88% at 5-85% RH and of about 0.85% at 85-95% RH.

The crystalline form of the besylate salt of cis-N-cyclohexyl-N-ethyl-3-(3-chloro-4-cyclohexylphenyl)prop-2-enylamine is an orthorhombic unit cell, space group Pna2₁, with unit cell parameters:

a=14.404 Å α=90°
b=40.782 Å β=90°
c=10.996 Å γ=90°
unit cell volume=6,459.6 Å³

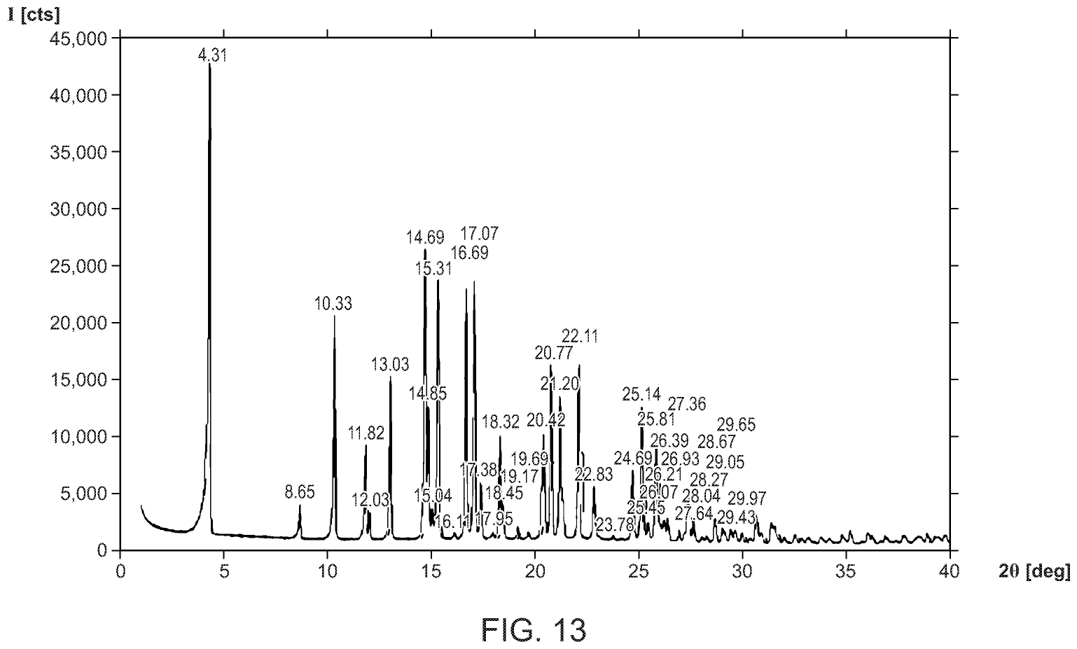

The XRPD patterns of the crystalline form of xinafoate salt of cis-N-cyclohexyl-N-ethyl-3-(3-chloro-4-cyclohexylphenyl)prop-2-enylamine is depicted in FIG. 13, and the peaks are tabulated in Table 6.

TABLE 6

| °2θ | d space (Å) | Intensity (%) |
|---|---|---|
| 4.31 ± | 20.473 ± | 100 |
| 0.20 | 0.949 | |
| 8.65 ± | 10.214 ± | 9 |
| 0.20 | 0.236 | |
| 10.33 ± | 8.553 ± | 48 |
| 0.20 | 0.165 | |
| 11.82 ± | 7.483 ± | 22 |
| 0.20 | 0.126 | |
| 12.03 ± | 7.349 ± | 8 |
| 0.20 | 0.122 | |
| 13.03 ± | 6.790 ± | 35 |
| 0.20 | 0.104 | |
| 14.69 ± | 6.026 ± | 61 |
| 0.20 | 0.082 | |
| 14.85 ± | 5.962 ± | 29 |
| 0.20 | 0.080 | |
| 15.04 ± | 5.887 ± | 8 |
| 0.20 | 0.078 | |
| 15.31 ± | 5.781 ± | 56 |
| 0.20 | 0.075 | |
| 16.11 ± | 5.498 ± | 4 |
| 0.20 | 0.068 | |
| 16.69 ± | 5.309 ± | 53 |
| 0.20 | 0.063 | |
| 17.07 ± | 5.189 ± | 55 |
| 0.20 | 0.060 | |
| 17.38 ± | 5.099 ± | 14 |
| 0.20 | 0.058 | |
| 17.95 ± | 4.938 ± | 4 |
| 0.20 | 0.055 | |
| 18.32 ± | 4.840 ± | 24 |
| 0.20 | 0.052 | |
| 18.45 ± | 4.805 ± | 9 |
| 0.20 | 0.052 | |
| 19.17 ± | 4.627 ± | 5 |
| 0.20 | 0.048 | |
| 19.69 ± | 4.506 ± | 4 |
| 0.20 | 0.045 | |
| 20.42 ± | 4.346 ± | 24 |
| 0.20 | 0.042 | |
| 20.77 ± | 4.273 ± | 38 |
| 0.20 | 0.041 | |
| 21.20 ± | 4.188 ± | 32 |
| 0.20 | 0.039 | |
| 22.11 ± | 4.017 ± | 38 |
| 0.20 | 0.036 | |
| 22.83 ± | 3.891 ± | 13 |
| 0.20 | 0.034 | |
| 23.78 ± | 3.739 ± | 3 |
| 0.20 | 0.031 | |
| 24.69 ± | 3.603 ± | 17 |
| 0.20 | 0.029 | |
| 25.14 ± | 3.539 ± | 29 |
| 0.20 | 0.028 | |
| 25.45 ± | 3.497 ± | 6 |
| 0.20 | 0.027 | |
| 25.81 ± | 3.449 ± | 24 |
| 0.20 | 0.026 | |
| 26.07 ± | 3.416 ± | 5 |
| 0.20 | 0.026 | |
| 26.21 ± | 3.398 ± | 6 |
| 0.20 | 0.025 | |
| 26.39 ± | 3.375 ± | 7 |
| 0.20 | 0.025 | |
| 26.93 ± | 3.308 ± | 4 |
| 0.20 | 0.024 | |
| 27.36 ± | 3.257 ± | 13 |
| 0.20 | 0.023 | |
| 27.64 ± | 3.225 ± | 5 |
| 0.20 | 0.023 | |
| 28.04 ± | 3.179 ± | 3 |
| 0.20 | 0.022 | |
| 28.27 ± | 3.154 ± | 3 |
| 0.20 | 0.022 | |
| 28.67 ± | 3.111 ± | 6 |
| 0.20 | 0.021 | |
| 29.05 ± | 3.071 ± | 4 |
| 0.20 | 0.021 | |
| 29.43 ± | 3.033 ± | 4 |
| 0.20 | 0.020 | |
| 29.65 ± | 3.011 ± | 4 |
| 0.20 | 0.020 | |
| 29.97 ± | 2.979 ± | 4 |
| 0.20 | 0.019 | |

### Example 5—Edisylate Salt of Cis-N-cyclohexyl-N-ethyl-3-(3-chloro-4-cyclohexylphenyl)prop-2-enylamine

Method 1: cis-N-cyclohexyl-N-ethyl-3-(3-chloro-4-cyclohexylphenyl)prop-2-enylamine (104.5 mg) was dissolved in a 50/50 v/v mixture of EtOAc with heptane (1 mL). Solids of ethanedisulfonic acid dihydrate (28.7 mg) were added to the solution. Formation of solids was observed. The mixture was stirred at about 55° C. for approximately 5 days, followed by stirring at ambient temperature for approximately 1 day. Solids were isolated by vacuum filtration. Removal of residual solvent under high vacuum yielded the salt.

Method 2 cis-N-cyclohexyl-N-ethyl-3-(3-chloro-4-cyclohexylphenyl)prop-2-enylamine (105.2 mg) was dissolved in anhydrous MTBE (1 mL). Solids of ethanedisulfonic acid dihydrate (30.8 mg) were added to the solution. Using a hotplate equipped with a magnetic stirrer, the mixture was stirred at approximately 45° C. for approximately 3 days followed by an ambient temperature stirring for approximately 3 days. Solids were isolated by vacuum filtration and were stored over desiccant. Removal of residual solvent under high vacuum yielded the salt. The XRPD pattern was consistent with the edisylate salt obtained by Method 1, possibly containing trace amount of free ethanedisulfonic acid.

The TGA trace of the crystalline form of edysilate salt of cis-N-cyclohexyl-N-ethyl-3-(3-chloro-4-cyclohexylphenyl)prop-2-enylamine is depicted in FIG. 14. Weight losses of approximately 2.5% from 38° C. to 120° C. and approximately 1.6% from 120° C. to 193° C. were observed in the TG thermogram. Large weight loss with onset at 260° C. corresponds to decomposition.

The DSC trace of the crystalline form of the crystalline form of edysilate salt of cis-N-cyclohexyl-N-ethyl-3-(3-chloro-4-cyclohexylphenyl)prop-2-enylamine is depicted in FIG. 15. The DSC thermogram displays a small endothermic peak at 119° C.

The DVS pattern of the crystalline form of the crystalline form of edysilate salt of cis-N-cyclohexyl-N-ethyl-3-(3-chloro-4-cyclohexylphenyl)prop-2-enylamine is depicted in FIG. 16. The DVS pattern displays a weight gain of about 0.35% at 5-75% RH and of about 18.55% at 75-95% RH.

The XRPD patterns of the crystalline form of edysilate salt of cis-N-cyclohexyl-N-ethyl-3-(3-chloro-4-cyclohexylphenyl)prop-2-enylamine is depicted in FIG. 13, and the peaks are tabulated in Table 7.

TABLE 7

| °2θ | d space (Å) | Intensity (%) |
|---|---|---|
| 5.14 ± | 17.189 ± | 24 |
| 0.20 | 0.669 | |
| 5.51 ± | 16.026 ± | 100 |
| 0.20 | 0.581 | |
| 6.12 ± | 14.422 ± | 23 |
| 0.20 | 0.471 | |
| 6.31 ± | 14.003 ± | 18 |
| 0.20 | 0.444 | |
| 8.37 ± | 10.551 ± | 3 |
| 0.20 | 0.252 | |
| 8.70 ± | 10.153 ± | 7 |
| 0.20 | 0.233 | |
| 9.24 ± | 9.563 ± | 5 |
| 0.20 | 0.207 | |
| 9.49 ± | 9.308 ± | 4 |
| 0.20 | 0.196 | |
| 9.82 ± | 9.002 ± | 4 |
| 0.20 | 0.183 | |
| 9.91 ± | 8.916 ± | 3 |
| 0.20 | 0.179 | |
| 10.06 ± | 8.785 ± | 4 |
| 0.20 | 0.174 | |
| 10.16 ± | 8.701 ± | 5 |
| 0.20 | 0.171 | |
| 10.29 ± | 8.590 ± | 4 |
| 0.20 | 0.167 | |
| 10.51 ± | 8.414 ± | 5 |
| 0.20 | 0.160 | |
| 11.03 ± | 8.013 ± | 20 |
| 0.20 | 0.145 | |
| 11.17 ± | 7.914 ± | 20 |
| 0.20 | 0.141 | |
| 12.50 ± | 7.076 ± | 3 |
| 0.20 | 0.113 | |
| 13.10 ± | 6.751 ± | 6 |
| 0.20 | 0.103 | |
| 13.22 ± | 6.694 ± | 9 |
| 0.20 | 0.101 | |
| 13.53 ± | 6.540 ± | 4 |
| 0.20 | 0.096 | |
| 13.67 ± | 6.474 ± | 9 |
| 0.20 | 0.094 | |
| 13.98 ± | 6.328 ± | 8 |
| 0.20 | 0.090 | |
| 14.07 ± | 6.291 ± | 8 |
| 0.20 | 0.089 | |
| 14.43 ± | 6.134 ± | 4 |
| 0.20 | 0.085 | |
| 14.59 ± | 6.066 ± | 6 |
| 0.20 | 0.083 | |
| 14.72 ± | 6.012 ± | 5 |
| 0.20 | 0.081 | |
| 15.05 ± | 5.880 ± | 7 |
| 0.20 | 0.078 | |
| 15.35 ± | 5.767 ± | 13 |
| 0.20 | 0.075 | |
| 15.45 ± | 5.731 ± | 12 |
| 0.20 | 0.074 | |
| 15.65 ± | 5.659 ± | 7 |
| 0.20 | 0.072 | |
| 16.03 ± | 5.526 ± | 7 |
| 0.20 | 0.069 | |
| 16.19 ± | 5.470 ± | 9 |
| 0.20 | 0.067 | |
| 16.35 ± | 5.417 ± | 15 |
| 0.20 | 0.066 | |
| 16.59 ± | 5.338 ± | 12 |
| 0.20 | 0.064 | |
| 16.81 ± | 5.269 ± | 5 |
| 0.20 | 0.062 | |
| 17.06 ± | 5.195 ± | 20 |
| 0.20 | 0.060 | |
| 17.56 ± | 5.046 ± | 17 |
| 0.20 | 0.057 | |
| 17.85 ± | 4.966 ± | 11 |
| 0.20 | 0.055 | |
| 18.13 ± | 4.888 ± | 10 |
| 0.20 | 0.053 | |
| 18.27 ± | 4.851 ± | 8 |
| 0.20 | 0.053 | |
| 18.55 ± | 4.780 ± | 12 |
| 0.20 | 0.051 | |
| 18.78 ± | 4.722 ± | 11 |
| 0.20 | 0.050 | |
| 18.91 ± | 4.690 ± | 13 |
| 0.20 | 0.049 | |
| 19.01 ± | 4.665 ± | 17 |
| 0.20 | 0.049 | |
| 19.23 ± | 4.612 ± | 5 |
| 0.20 | 0.048 | |
| 19.46 ± | 4.557 ± | 13 |
| 0.20 | 0.046 | |
| 19.72 ± | 4.498 ± | 12 |
| 0.20 | 0.045 | |
| 19.88 ± | 4.463 ± | 17 |
| 0.20 | 0.044 | |
| 20.21 ± | 4.391 ± | 8 |
| 0.20 | 0.043 | |
| 20.64 ± | 4.300 ± | 20 |
| 0.20 | 0.041 | |
| 21.12 ± | 4.203 ± | 15 |
| 0.20 | 0.039 | |
| 21.31 ± | 4.166 ± | 9 |
| 0.20 | 0.039 | |
| 21.58 ± | 4.114 ± | 15 |
| 0.20 | 0.038 | |
| 22.24 ± | 3.994 ± | 12 |
| 0.20 | 0.035 | |
| 22.48 ± | 3.953 ± | 13 |
| 0.20 | 0.035 | |
| 23.12 ± | 3.845 ± | 15 |
| 0.20 | 0.033 | |
| 23.50 ± | 3.782 ± | 8 |
| 0.20 | 0.032 | |
| 23.77 ± | 3.740 ± | 11 |
| 0.20 | 0.031 | |
| 24.09 ± | 3.692 ± | 11 |
| 0.20 | 0.030 | |
| 24.63 ± | 3.612 ± | 12 |
| 0.20 | 0.029 | |
| 24.94 ± | 3.568 ± | 10 |
| 0.20 | 0.028 | |
| 25.18 ± | 3.533 ± | 9 |
| 0.20 | 0.028 | |
| 25.77 ± | 3.454 ± | 7 |
| 0.20 | 0.026 | |
| 26.15 ± | 3.405 ± | 7 |
| 0.20 | 0.026 | |
| 26.63 ± | 3.345 ± | 8 |
| 0.20 | 0.025 | |
| 27.28 ± | 3.267 ± | 5 |
| 0.20 | 0.023 | |

### Example 6—Determination of Aqueous Kinetic Solubility of the Selected Salts

Kinetic solubility was estimated by aliquot addition method, and dissolution was judged by visual observations. If dissolution was not observed, values are reported as “less than.” If complete dissolution was observed upon the first aliquot of solvent, values are reported as “greater than.” Kinetic solubility of selected salts is presented in Table 8.

TABLE 8

| | Kinetic |
|---|---|
| | Solubility, |
| Salt | mg/mL |
| Esylate salt | 30 |
| Salicylate salt | <0.9 |
| Besylate salt | <0.6 |
| Xinafoate salt | <0.7 |
| HCl salt | <0.9 |

Data shown in Table 8 indicate that while salicylate, besylate, and xinafoate of cis-N-cyclohexyl-N-ethyl-3-(3-chloro-4-cyclohexylphenyl)prop-2-enylamine display kinetic solubility similar to that of the HCl salt, the esylate salt is unexpectedly more than 30 fold more soluble than all other crystalline salts of cis-N-cyclohexyl-N-ethyl-3-(3-chloro-4-cyclohexylphenyl)prop-2-enylamine isolated.

### Methods of Compound Testing

Mouse OPC preparation: To rigorously assess effects of treatments on OPCs, all compounds can be assayed in two independent platings of epiblast stem cell-derived OPCs. OPCs can be generated from EpiSC5 line of mouse epiblast stem cells. EpiSC-derived OPCs can be obtained using in vitro differentiation protocols and culture conditions described previously (Najm, F. J. et al (2011) “Rapid and robust generation of functional oligodendrocyte progenitor cells from epiblast stem cells,” Nature Methods 8(11):957-962). To ensure uniformity throughout all in vitro screening experiments, EpiSC-derived OPCs can be sorted to purity by fluorescence activated cell sorting at passage four with conjugated CD 140α-APC (eBioscience, 17-1401; 1:80). Sorted batches of OPCs can be expanded and frozen down in aliquots. OPCs can be thawed into growth conditions for at least one passage before use in further assays.

In vitro phenotypic screening of OPCs: EpiSC-derived OPCs can be grown and can be expanded in poly-L-ornithine (PO) and laminin-coated flasks in N2B27 media (DMEM/F12 (Gibco), N2-MAX (R&D Systems), B-27 (ThermoFisher), and GlutaMax (Gibco)) supplemented with FGF2 (10 μg/mL, R&D systems, 233-FB-025) and PDGF-AA (10 μg/mL, R&D systems, 233-AA-050) before harvesting for experiments. The cells can be seeded onto poly-L-ornithine or poly-D-lysine coated 96-well CellCarrier Ultra plates (PerkinElmer) coated with laminin (Sigma, L2020) using a multi-channel pipet. Fifty-thousand cells can be seeded per well in media containing N2 and B27 without growth factors, and can be allowed to attach for 30 min before addition of drug. For dose-response testing of hits, a 1000× compound stock in dimethyl sulphoxide (DMSO) can be added to assay plates with 0.1 μL solid pin multi-blot replicators (V & P Scientific; VP 409), resulting in a final primary screening dose curve of 8 doses between 1000 nM and 0.5 nM. Positive controls and DMSO vehicle controls can be included in each assay plate. Cells can be incubated under standard conditions (37° C., 5% CO₂) for 3 days and fixed with 4% paraformaldehyde (PFA) in phosphate buffered saline (PBS) for 20 min. Fixed plates can be washed with PBS (200 μL per well) twice, permeabilized with 0.1% Triton X-100 and blocked with 10% donkey serum (v/v) in PBS for 40 min. Then, cells can be labelled with MBP antibodies (Abcam, ab7349; 1:200) for 1 h at room temperature followed by detection with Alexa Fluor conjugated secondary antibodies (1:500) for 45 min. Nuclei can be visualized by DAPI staining (Sigma; 1 μg/ml). During washing steps, PBS can be added using a multi-channel pipet and aspiration can be performed using Biotek EL406 washer dispenser (Biotek) equipped with a 96-well aspiration manifold.

High-content imaging and analysis: Plates can be imaged on the Operetta High Content Imaging and Analysis system (PerkinElmer) and a set of 6 fields can be captured from each well. Analysis (PerkinElmer Harmony and Columbus software) can begin by identifying intact nuclei stained by DAPI; that is, those traced nuclei that can be larger than 300 μm² in surface area. Each traced nucleus region can then be expanded by 50% and cross-referenced with the mature myelin protein (MBP) stain to identify oligodendrocyte nuclei, and from this the percentage of oligodendrocytes can be calculated. EC50 values can be calculated using The Levenberg-Marquardt algorithm to fit a Hill equation to dose-response data (0.5 nM to 1000 nM).

GCMS-based sterol profiling: Sterols can be monitored using a modified Folch wash protocol (Hubler Z. et al (2018) “Accumulation of 8,9-unsaturated sterols drives oligodendrocyte formation and remyelination,” Nature 560(7718):372-376). EpiSC-derived OPCs can be plated at one million cells per well in PO- and laminin-coated six-well plates in N2B27 media without growth factors. After 24 hours, cells can be dissociated with Accutase, can be rinsed with saline, and cell pellets can be frozen. Cells can be lysed in methanol (Sigma-Aldrich) with agitation for 30 minutes and cell debris can be removed by centrifugation at 10,000 rpm for 15 min. Cholesterol-d7 standard (Cambridge Isotope Laboratories) can be added before drying under nitrogen stream and derivatization with 55 μl of bis(trimethylsilyl) trifluoroacetamide. After derivatization, 1 μl can be analyzed by gas chromatography/mass spectrometry using an Agilent 5973 Network Mass Selective Detector equipped with a 6890 gas chromatograph system and a HP-5MS capillary column (60 m×0.25 mm×0.25 mm). Samples can be analyzed in full scan mode using electron impact ionization; ion fragment peaks can be integrated to calculate sterol abundance, and quantitation can be relative to cholesterol-d7. The following ion fragments can be used to quantitate each metabolite: cholesterol-d7 (465), FF-Mas (482), cholesterol (368), zymostenol (458), zymosterol (456), Desmosterol (456, 343), 7-dehydrocholesterol (456, 325), lanosterol (393), lathosterol (458), 14-dehydrozymostenol (456, 351). All standards can be obtained from Avanti Polar Lipids unless otherwise indicated. Calibration curves can be generated by injecting varying concentrations of sterol standards and maintaining a fixed amount of cholesterol-D7.

| Analyte increased | Cholesterol enzyme inhibited |
|---|---|
| Lanosterol | CYP51 |
| FF-MAS | TM7SF2 (Sterol 14 Reductase) |
| 14-dehydrozymostenol | TM7SF2 (Sterol 14 Reductase) |
| Zymosterol | EBP |
| Zymostenol | EBP |
| 7-dehydrocholesterol | DHCR7 |

Table 9. Sterol GC-MS analytes and their relationship with inhibitors of cholesterol biosynthesis

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

What is claimed is:

1. A solid form of the compound represented by formula (II):

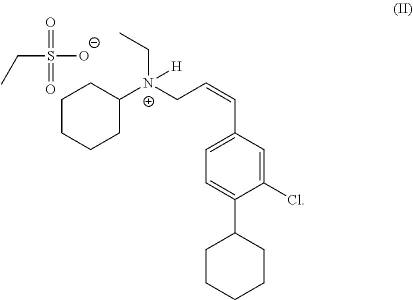

2. The solid form of claim 1, wherein the solid form is an amorphous, polymorphous, crystalline, or partially crystalline solid form.

3. The solid form of claims 1 or 2, wherein the solid form is a crystalline form characterized by at least three x-ray powder diffraction peaks at 2θ angles selected from 12.50°, 16.73°, 17.87°, and 20.47°.

4. The crystalline form of claim 3, wherein the crystalline form is characterized by at least four x-ray powder diffraction peaks at 2θ angles selected from 12.50°, 14.00°, 16.73°, 17.87°, 18.65°, and 20.47°.

5. The crystalline form of claim 3, wherein the crystalline form is characterized by at least five x-ray powder diffraction peaks at 2θ angles selected from 6.97°, 12.50°, 14.00°, 14.75°, 16.73°, 17.87°, 18.65°, 18.98°, and 20.47°.

6. The crystalline form of claim 3, wherein the crystalline form is characterized by x-ray powder diffraction peaks at 2θ angles of 12.50°, 16.73°, 17.87°, and 20.47°.

7. The crystalline form of claim 3, wherein the crystalline form is characterized by x-ray powder diffraction peaks at 2θ angles of 12.50°, 14.00°, 16.73°, 17.87°, 18.65°, and 20.47°.

8. The crystalline form of claim 3, wherein the crystalline form is characterized by x-ray powder diffraction peaks at 2θ angles of 6.97°, 12.50°, 14.00°, 14.75°, 16.73°, 17.87°, 18.65°, 18.98°, and 20.47°.

9. The crystalline form of any one of claims 3-8, wherein the crystalline form is characterized by an x-ray powder diffraction pattern substantially in accordance with that depicted in FIG. 1.

10. The crystalline form of any of claims 3-9, wherein the crystalline form is characterized by a DSC thermogram having an endothermic event at about 137° C.

11. The crystalline form of any of claims 3-9, wherein the crystalline form is characterized by a DSC thermogram substantially in accordance with that depicted in FIG. 3.

12. The solid form of any one of claims 1-11, wherein the solid form is substantially free of solvent.

13. The solid form of any one of claims 1-12, wherein the solid form is substantially free of water.

14. The solid form of any one of claims 1-13, wherein the solid form is anhydrous.

15. The solid form of any one of claims 1-14, wherein the solid form is substantially pure.

16. The solid form of any one of claims 1-15, wherein the solid form is substantially free of chemical impurities.

17. The solid form of any one of claims 1-16, wherein the solid form is substantially free of physical impurities.

18. The solid form of any one of claims 1-11, wherein the solid form is a solvate.

19. The solid form of any one of the claims 1-11 and 18, wherein the solid form is a hydrate.

20. A pharmaceutical composition, comprising the solid form of any one of claims 1-19 and a pharmaceutically acceptable carrier.

21. A solid form of the compound of formula (III):

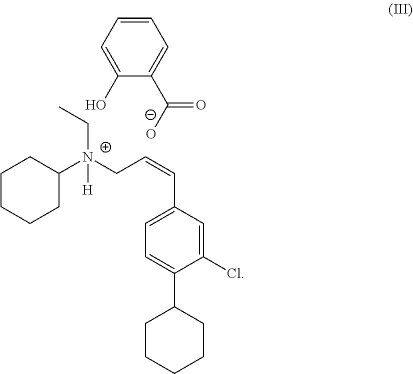 [SMILES: CC[NH+](C/C=C\c1ccc(C2CCCCC2)c(Cl)c1)C1CCCCC1 ; O=C([O-])c1ccccc1O]

22. The solid form of claim 21, wherein the solid form is an amorphous, polymorphous, crystalline, or partially crystalline solid form.

23. The solid form of claims 21 or 22, wherein the solid form is a crystalline form characterized by at least three x-ray powder diffraction peaks at 2θ angles selected from 4.93°, 14.71°, 20.85°, and 25.39°.

24. The crystalline form of claim 23, wherein the crystalline form is characterized by at least four x-ray powder diffraction peaks at 2θ angles selected from 4.93°, 14.71°, 19.03°, 20.85°, 24.94°, and 25.39°.

25. The crystalline form of claim 23, wherein the crystalline form is characterized by at least five x-ray powder diffraction peaks at 2θ angles selected from 4.93°, 12.79°, 14.71°, 16.97°, 17.59°, 19.03°, 20.85°, 22.08°, 24.94°, and 25.39°.

26. The crystalline form of claim 23, wherein the crystalline form is characterized by x-ray powder diffraction peaks at 2θ angles of 4.93°, 14.71°, 20.85°, and 25.39°.

27. The crystalline form of claim 23, wherein the crystalline form is characterized by x-ray powder diffraction peaks at 2θ angles of 4.93°, 14.71°, 19.03°, 20.85°, 24.94°, and 25.39°.

28. The crystalline form of claim 23, wherein the crystalline form is characterized by x-ray powder diffraction peaks at 2θ angles of 4.93°, 12.79°, 14.71°, 16.97°, 17.59°, 19.03°, 20.85°, 22.08°, 24.94°, and 25.39°.

29. The crystalline form of any of claims 23-28, wherein the crystalline form is characterized by an x-ray powder diffraction pattern substantially in accordance with that depicted in FIG. 5.

30. The crystalline form of any of claims 23-29, wherein the crystalline form is characterized by a DSC thermogram having an endothermic event at about 141° C.

31. The crystalline form of any of claims 23-29, wherein the crystalline form is characterized by a DSC thermogram substantially in accordance with that depicted in FIG. 7.

32. The solid form of any one of the claims 21-31, wherein the solid form is substantially free of solvent.

33. The solid form of any one of the claims 21-32, wherein the solid form is substantially free of water.

34. The solid form of any one of the claims 21-33, wherein the solid form is anhydrous.

35. The solid form of any one of the claims 21-34, wherein the solid form is substantially pure.

36. The solid form of any one of the claims 21-35, wherein the solid form is substantially free of chemical impurities.

37. The solid form of any one of the claims 21-36, wherein the solid form is substantially free of physical impurities

38. A pharmaceutical composition, comprising the solid form of any one of claims 21-37 and a pharmaceutically acceptable carrier.

39. A method of promoting myelination of central nervous system neurons in a subject suffering from a myelin-related disorder, the method comprising administering to the subject a therapeutically effective amount of the solid form of any one of claims 1-19 and 21-37 or a pharmaceutical composition of claims 20 or 38.

40. The method of claim 39, wherein the myelin-related disorder is selected from multiple sclerosis (MS), neuromyelitis optica (NMO), optic neuritis, pediatric leukodystrophies, neonatal white matter injury, age-related dementia, schizophrenia, progressive multifocal leukoencephalopathy (PML), encephalomyelitis (EPL), central pontine myelolysis (CPM), adrenoleukodystrophy, Alexander's disease, Pelizaeus Merzbacher disease (PMD), Vanishing White Matter Disease, Wallerian Degeneration, transverse myelitis, amylotrophic lateral sclerosis (ALS), Huntington's disease, Alzheimer's disease, Parkinson's disease, spinal cord injury, traumatic brain injury, post radiation injury, neurologic complications of chemotherapy, stroke, acute ischemic optic neuropathy, vitamin E deficiency, isolated vitamin E deficiency syndrome, Bassen-Kornzweig syndrome, Marchiafava-Bignami syndrome, metachromatic leukodystrophy, trigeminal neuralgia, acute disseminated encephalitis, Guillian-Barre syndrome, Charcot-Marie-Tooth disease, Bell's palsy, and radiation-induced demyelination.

41. The method of claim 40, wherein the disorder is selected from: neuromyelitis optica (NMO), optic neuritis, pediatric leukodystrophies, neonatal white matter injury, age-related dementia, and schizophrenia.

42. The method of claim 40, wherein the subject is suffering from multiple sclerosis.

43. The method of claim 42, wherein the multiple sclerosis is classified as primary progressive MS (PPMS).

44. The method of claim 42, wherein the multiple sclerosis is classified as relapsing and remitting MS (RRMS).

45. The method of claim 42, wherein the multiple sclerosis is classified as secondary progressive MS (SPMS).

46. The method of claim 42, wherein the subject is a human.

47. The method of claim 46, wherein the human is a female.

48. The method of claim 39, wherein the neurons are in the brain, spinal cord, or both the brain and spinal cord.

49. The method of any one of claims 39-48, wherein the pharmaceutical composition is administered intravenously, intrathecally, subcutaneously, intramuscularly, intranasally or orally.

50. The method of any one of claims 42-47, further comprising administering a therapeutically effective amount of an MS therapeutic agent.

51. The method of claim 50, wherein the MS therapeutic agent is selected from: glatiramer acetate, Ocrevus (ocrelizumab), Campath (Lemtrada or alemtuzumab), Gilenya, Ampyra (dalfampridine), Tysabri (natalizumab), Aubagio (teriflunomide), Rebif, Avonex, Betaseron, Plegridy, Interferon Beta-la, dimethyl fumarate, fingolimod, rituximab, Zinbryta, Ofatumymab, Nerventra (laquinimod), Masitinib, Siponimod, Ozanimod, Ponesimod, ibudilast, vatelizumab, minocycline, ibrutinib, PRN2246, Cladripine, GNBAC1, daclizumab, and MD1003 (biotin).

52. The method of claim 50, wherein the MS therapeutic agent is administered simultaneously with the solid form of any one of claims 1-19 and 21-37 or a pharmaceutical composition of claims 20 or 38.

53. The method of claim 50, wherein the MS therapeutic agent is administered prior to administration the solid form of any one of claims 1-19 and 21-37 or a pharmaceutical composition of claims 20 or 38.

54. The method of claim 50, wherein the MS therapeutic agent is administered following the administration of the solid form of any one of claims 1-19 and 21-37 or a pharmaceutical composition of claims 20 or 38.

55. The method of any one of claims 39-54, wherein the subject is administered the compound for an on-drug cycle of at least three months.

56. The method of claim 55, wherein the subject is administered the compound for an on-drug cycle of at least six months.

57. The method of any one of claims 39-54, wherein the subject is administered the solid form of any one of claims 1-19 and 21-37 or a pharmaceutical composition of claims 20 or 38 using the following dosing regimen:
c. on-drug cycle for at least six months;
d. off-drug cycle for at least three months;
wherein the on-drug and off-drug cycles are optionally repeated.

58. The method of any one of claims 39-57, wherein the compound inhibits enzyme mediate synthesis of one or more sterol intermediates in the cholesterol biosynthesis pathway.

59. The method of any one of claims 39-58, wherein the compound promotes accumulation of Δ8,9-unsaturated sterol intermediates in the cholesterol biosynthesis pathway.

60. The method of any one claims 39-57, wherein the compound inhibits one or more of CYP51, sterol-14-reductase, or EBP enzyme mediated synthesis of sterol intermediates in the cholesterol biosynthesis pathway.

61. The method of any one claims 39-57, wherein the compound induces, promotes, and/or modulates oligodendrocyte precursor cell (OPC) differentiation, proliferation and/or maturation.

62. The method of claim 61, wherein the induction of OPC differentiation is characterized by an increase in myelin basic protein (MBP) expression.

63. A method of inducing endogenous oligodendrocyte precursor cell (OPC) differentiation in a subject in need thereof, the method comprising administering to the subject a therapeutically effective amount of the solid form of any one of claims 1-19 and 21-37 or a pharmaceutical composition of claims 20 or 38.

64. The method of claim 63, wherein the subject suffers from a myelin-related disorder.

65. The method of claim 64, wherein the subject is suffering from multiple sclerosis.

66. The method of any one of claims 63-65, wherein the subject is human.

67. The method of any one of claims 63-66, further comprising administering a therapeutically effective amount of an MS therapeutic agent.
